(19) United States
(12) Patent Application Publication
(10) Pub. No.: US 2017/0071591 A1
(43) Pub. Date: Mar. 16, 2017

(54) DEVICES AND METHODS FOR CLOSING OPENINGS IN BODY TISSUE

(71) Applicant: SATURIX LTD., Tel-Aviv (IL)

(72) Inventors: Zvi GOLTZMAN, Rosh-ha'ayin (IL); Yehuda BACHAR, Givaat Shmuel (IL)

(21) Appl. No.: 15/121,933

(22) PCT Filed: Feb. 26, 2015
(86) PCT No.: PCT/IB2015/051441

§ 371 Date: Aug. 26, 2016

Related U.S. Application Data
(60) Provisional application No. 61/946,193, filed on Feb. 28, 2014.

(51) Int. Cl. A61B 17/04; A61B 17/062; A61B 17/29; A61B 17/06

(52) U.S. Cl. CPC A61B 17/0401; A61B 17/06066; A61B 17/0625; A61B 17/29; A61B 2017/0409; A61B 2017/0414; A61B 2017/0464; A61B 2017/0445; A61B 2017/0454; A61B 2017/06095; A61B 2017/2926

(57) ABSTRACT

Devices and methods for closing openings in body tissue. Includes a delivery device for delivering a body tissue connecting device; a body tissue connecting device; methods using thereof; tissue connecting elements (for example, as a form of tissue suture anchors); and applications thereof, such as for closing openings in body tissue, for example, associated with cuts, incisions, or other soft tissue discontinuities.

### RELATED APPLICATIONS

The present application claims the benefit under 35 U.S.C. 119(e) of U.S. Provisional Application 61/946,193 filed on Feb. 28, 2014, the disclosure of which is incorporated herein by reference.

### FIELD OF THE INVENTION

The present invention, in some embodiments thereof, relates to devices and methods for closing openings in body tissue. Some embodiments of the present invention particularly relate to a delivery device for delivering a body tissue connecting device; a body tissue connecting device, methods using thereof; tissue connecting elements (for example, as a form of tissue suture anchors); and applications thereof, such as for closing openings in body tissue, for example, associated with cuts, incisions, or other soft tissue discontinuities.

### BACKGROUND OF THE INVENTION

Devices and methods employed for closing openings in body tissue, for example, using tissue suture anchors, are known in the art. Exemplary teachings of such are disclosed in U.S. Pat. No. 5,464,426; and in U.S. Patent Application Publication Nos. US 2005/0113851 and US 2007/0088390. Basically, tissue suture anchors are first deployed around the boundary of the body opening, commonly readily threaded with a suture, and then the suture is pulled to bring the threaded anchors closer together while closing the body opening.

Additional exemplary teachings of such techniques are disclosed in same applicant/assignee PCT International Patent Application Publication No. WO 2014/033692 A2, describing an apparatus, and a suture anchor, for securing a suture thread to a region of tissue.

### SUMMARY OF THE INVENTION

The present invention, in some embodiments thereof, relates to devices and methods for closing openings in body tissue. Some embodiments of the present invention particularly relate to a delivery device for delivering a body tissue connecting device; a body tissue connecting device; methods using thereof; tissue connecting elements (for example, as a form of tissue suture anchors); and applications thereof, such as for closing openings in body tissue, for example, associated with cuts, incisions, or other soft tissue discontinuities.

According to an aspect of some embodiments of the present invention, there is provided a delivery device for delivering a body tissue connecting device, the delivery device comprising: a shaft connected at a distal end thereof to a first jaw, both sharing a shaft lumen extending lengthwise therein and opened at a distal end of the first jaw; a needle, comprising a needle lumen and a needle distal tip, extending inside the shaft lumen and slidable therein to a protruding position, in which the needle distal tip protrudes through the first jaw distal end at a pre-determined maximal protrusion length, and to a retracted position, in which the needle lumen is sized to house a plurality of tissue connecting elements strung on a thread; and a second jaw pivotally connected at the shaft distal end or/and to the first jaw, and linked to a jaw actuator configured to selectively actuate the second jaw between a closed position, whereby the second jaw is in close approximation with the first jaw, and an opened position, whereby the second jaw is pivoted away from the first jaw; and a holder mechanism provided in a distal end of the second jaw and configured to arrest a distal-most tissue connecting element to the second jaw when the second jaw is in the closed position and when the distal-most tissue connecting element partially protrudes from the first jaw distal end while the needle is in the retracted position thereof.

According to some embodiments of the invention, the needle lumen includes the thread, the thread being connected at a distal end thereof to a stop element and the tissue connecting elements strung proximally to the stop element and compactable along the thread towards the stop element.

According to some embodiments of the invention, the delivery device further comprises a pusher having a pusher distal end sized to move distally inside of the needle lumen so as to force the tissue connecting elements to compact together or/and shift towards the needle distal tip.

According to some embodiments of the invention, the jaws are sized, shaped and arranged to grasp a body tissue having a thickness within a range of pre-determined allowed thickness values.

According to some embodiments of the invention, the second jaw includes an entrapment opening sized to allow the needle to pass therethrough, such that upon the second jaw grasping the body tissue the needle distal tip, when protruded to the maximal protrusion length, passes through the entrapment opening while penetrating through the body tissue.

According to some embodiments of the invention, the entrapment opening is sized to allow the needle to pass therethrough via an acute angle relative to the second jaw.

According to some embodiments of the invention, the delivery device further comprises a restraint configured to restrain any of the tissue connecting elements from passing distally therethrough, when the tissue connecting elements are subjected to a pushing force less than or equal to a maximal restraining force.

According to some embodiments of the invention, the tissue connecting elements are compactable to a selected configuration when pressed against the restraint with the pushing force less than or equal to the maximal restraining force.

According to some embodiments of the invention, the holder mechanism is configured to apply a coupling force greater than the maximal restraining force so that the distal-most tissue connecting element is releasable from the restraint upon the second jaw shifting from the closed position to the opened position with the distal-most tissue connecting element being arrested to the second jaw.

According to some embodiments of the invention, the restraint is located such that upon partial protrusion of the distal-most tissue connecting element through the shaft distal end, the restraint restrains the tissue connecting element located immediately following the distal-most tissue connecting element.

According to some embodiments of the invention, the restraint includes, or is in a form of, a curvature in the needle lumen and surrounding wall of the needle, whereby any of the tissue connecting elements forced therethrough will bend in conformity to the curvature.

According to some embodiments of the invention, the restraint includes, or is in a form of, bulges or/and slits across surrounding wall of the needle, to thereby increase resistance to motion of any of the tissue connecting elements forced thereacross.

According to some embodiments of the invention, the delivery device further comprises a thread tensioner configured to apply tension to the thread extending between the tensioner and the stop element.

According to some embodiments of the invention, the thread tensioner includes a spring configured to apply a continuous tensioning force to the thread while allowing the thread to extend to a longer length between the tensioner and the stop element when the thread is pulled distally under a pulling force greater than a maximal tensioning force applied by the thread tensioner.

According to some embodiments of the invention, the tensioner lock configured to lock the thread at a selected extension length between the stop element and the thread tensioner.

According to some embodiments of the invention, the shaft lumen portion extending across within the first jaw includes a curved section, to thereby angularly force the needle therethrough such that the needle distal tip angles towards the second jaw.

According to some embodiments of the invention, the needle distal tip angles to an acute angle relative to a longitudinal axis of the shaft.

According to some embodiments of the invention, the needle includes a flexible portion.

According to some embodiments of the invention, the flexible portion includes a number of proximate transverse cuts.

According to some embodiments of the invention, the needle includes a rigid portion distal to and adjacent the flexible portion.

According to some embodiments of the invention, the rigid portion has length and shape matching a length or/and a shape of the curved section of the shaft lumen.

According to some embodiments of the invention, the at least one of the tissue connecting elements includes a tubular wall extending between opposing ends, each of the opposing ends merges with a corresponding longitudinal opening, the longitudinal openings oppose each other and are separated with a strip, wherein the at least one tissue connecting element is configured and arranged such that upon the thread being pulled back, the thread portion strung therein shifts from extending between the opposing ends to wrapping around the strip.

According to some embodiments of the invention, the edges surrounding each of the longitudinal openings are outlined so as to allow the thread portion strung therein to unhinderedly slip thereon or/and pass above and towards the strip.

According to some embodiments of the invention, each of the opposing ends of the at least one tissue connecting element includes a contact surface having an area being at least half a cross section area of a tube having inner diameter and outer diameter same as of the tubular wall.

According to some embodiments of the invention, a perimeter enclosing the contact surface comprises an outer part and an inner part, the perimeter outer part is greater than half a circumference of the tube.

According to some embodiments of the invention, the perimeter inner part includes two symmetrically opposing tangential lines touching both ends of a curve having an inner diameter same as the tube inner diameter.

According to some embodiments of the invention, the holder mechanism includes a holder actuator configured to selectively operate a holding member between a holding configuration, in which one of the tissue connecting elements is arrestable to the second jaw, and a releasing configuration, in which a previously arrested tissue connecting element to the second jaw is released therefrom.

According to some embodiments of the invention, the holding member includes a gripping portion slidable across the entrapment opening between the releasing configuration, in which the gripping portion is positioned distally beyond the entrapment opening, and the holding configuration, in which the gripping portion approximates the entrapment opening proximal side in a distance equal to or less than outer diameter of the needle.

According to some embodiments of the invention, the gripping portion approximates the entrapment opening proximal side to a distance equal to or less than outer diameter of the distal-most tissue connecting element, to thereby arrest the distal-most tissue connecting element to the second jaw upon withdrawal of the needle to the shaft lumen.

According to some embodiments of the invention, the holder mechanism is configured to force the distal-most tissue connecting element to align normally to the second jaw upon withdrawal of the needle to the shaft lumen and to arrest the distal-most tissue connecting element to the second jaw.

According to some embodiments of the invention, the pusher comprises or is fixedly connected to a linear ratchet comprising a first line of spaced indentations, each of the indentations corresponds in length to one of the tissue connecting elements, such that the pusher is restricted from proximal progression and each sequenced distal progression thereof is configured to push a first one of the tissue connecting elements to an area in the shaft lumen previously occupied by a distally adjacent one of the tissue connecting elements of same length.

According to some embodiments of the invention, the pusher or the linear ratchet comprises a second line of spaced indentations, each of the spaced indentations corresponds to the pre-determined maximal protrusion length, such that the needle is slidable between the protruding position and the retracted position while the pusher is positioned inside of the shaft lumen.

According to some embodiments of the invention, the needle distal tip is pointed so as to penetrate through a body tissue having, or compressible to, a thickness equal to or less than the pre-determined maximal protrusion length.

According to some embodiments of the invention, the needle distal tip projects from a lower part of the needle distal end at which an upper part of the needle distal end is substantially flat.

According to an aspect of some embodiments of the present invention, there is provided a device for delivering a body tissue connecting device, the delivery device comprising: a shaft having a shaft distal end and a shaft lumen extending lengthwise therein and opened at the shaft distal end; a needle, comprising a needle lumen and a needle distal tip, extending inside the shaft lumen and slidable therein to a protruding position, in which the needle distal tip protrudes through the shaft distal end at a maximal protrusion length, and to a retracted position, wherein the needle lumen is sized to house a plurality of tissue connecting elements; and an arm member connected at the shaft distal end, and an arm actuator configured to selectively actuate the arm member between a closed position and an opened position, the arm member includes a holder mechanism configured to couple with a distal-most fastener when the arm is in the closed position and when the distal-most fastener protrudes partially through the shaft distal end while the needle is in the retracted position; wherein the needle includes a flexible portion along at least part of a distal end thereof.

According to some embodiments of the invention, the flexible portion includes a number of proximate transverse cuts.

According to an aspect of some embodiments of the present invention, there is provided a body tissue connecting device, for closing an opening in body tissue, the device comprising: a thread fixedly connected at a distal end thereof to a stop element; and a plurality of tissue connecting elements strung and freely slidable on the thread proximally to the stop element and compactable to an elongated separable shell, along the thread, upon compression thereof against the stop element; wherein each of the tissue connecting elements is sized and shaped to pass through a puncture in a body tissue wall when aligned towards the puncture and to revolve and press against the body tissue wall when pulled back via the thread.

According to some embodiments of the invention, the stop element is a distal-most component of the plurality of tissue connecting elements.

According to some embodiments of the invention, the stop element is a blocking member or a thread knotted portion having a diameter greater than an inner diameter of a distal-most element of the plurality of tissue connecting elements.

According to some embodiments of the invention, the needle lumen houses the tissue connecting elements and the tissue connecting elements are releaseably connected to each other, or/and the tissue connecting elements are unconnected to each other.

According to some embodiments of the invention, the device includes at least three of the tissue connecting elements, or includes at least nine of the tissue connecting elements.

According to some embodiments of the invention, the tissue connecting elements are elastic, semi-elastic or/and bendable.

According to some embodiments of the invention, at least one of the tissue connecting elements includes a tubular wall extending between opposing ends, each of the opposing ends merges with a corresponding longitudinal opening, the longitudinal openings oppose each other and are separated with a strip, wherein the at least one tissue connecting element is configured and arranged such that upon the thread being pulled back, the thread portion strung therein shifts from extending between the opposing ends to wrapping around the strip.

According to some embodiments of the invention, the edges surrounding each of the longitudinal openings are outlined so as to allow the thread portion strung therein to unhinderedly slip thereon or/and pass above and towards the strip.

According to some embodiments of the invention, each of the opposing ends of the at least one tissue connecting element includes a contact surface having an area being at least half a cross section area of a tube having inner diameter and outer diameter same as of the tubular wall.

According to some embodiments of the invention, a perimeter enclosing the contact surface comprises an outer part and an inner part, the perimeter outer part is greater than half a circumference of the tube.

According to some embodiments of the invention, the perimeter inner part includes two symmetrically opposing tangential lines touching both ends of a curve having an inner diameter same as the tube inner diameter.

According to some embodiments of the invention, the partitionable shell is gaplessly compactable to a closed tube form.

According to some embodiments of the invention, the device further comprises a pusher member readily strung on the thread proximally to the tissue connecting elements and selectively applicable to apply a compressive force to the tissue connecting elements.

According to some embodiments of the invention, the device further comprises a locking member applicable to lock the tissue connecting elements along a selected length of the thread between the locking member and the stop element.

According to some embodiments of the invention, each adjacent pair of the tissue connecting elements includes a first rotationally asymmetric tissue connecting element identical in shape and size to a second tissue connecting element, wherein the first tissue connecting element is rotatable thru a straight angle relative to the second tissue connecting element and around a shared longitudinal axis thereof.

According to an aspect of some embodiments of the present invention, there is provided a method for closing an opening in body tissue, the method comprising: providing a needle comprising a needle distal tip and a needle lumen opened at the distal needle tip, the needle lumen housing a thread, fixedly connected at a distal end thereof to a stop element, and a partitionable shell comprising a plurality of tissue connecting elements strung on the thread proximally to the stop element and compactable along the thread towards the stop element, wherein each of the tissue connecting elements includes a tubular wall extending between opposing ends, each of the opposing ends merges with a corresponding longitudinal opening, the longitudinal openings being opposed to each other and separated with a strip; tensioning the thread between the stop element and a point at the thread proximally to the partitionable shell, via applying a tensioning force to the thread; penetrating a wall portion of body tissue adjacent the body tissue opening with the needle distal tip such that the needle distal tip moves from pointing towards an external surface of the wall portion to protruding beyond an internal surface of the wall portion; positioning the tissue connecting element located distal-most along the partionable shell beyond the internal surface of the wall portion in the needle lumen; retracting the needle back from the wall portion while holding the distal-most tissue connecting element beyond the internal surface of the wall portion such that the needle exposes the distal-most tissue connecting element; and separating the distal-most tissue connecting element from the partionable shell and spacing therebetween.

According to some embodiments of the invention, pulling the thread proximally such that a thread portion located within the distal-most tissue connecting element shifts from extending between the opposing ends to wrapping around the strip followed by the distal-most tissue connecting element revolving and pressing against the wall portion at the internal surface thereof.

According to some embodiments of the invention, tensioning of the thread is continuously performed during the penetrating, the positioning, the retracting and the separating, and includes, or is included in, the pulling of the thread, such that the tensioning force facilitates or contributes to the pulling.

According to some embodiments of the invention, the method is repeated, each time with a new distal-most tissue connecting element consecutive to a previous distal-most tissue connecting element, until a selected number of the tissue connecting elements is separated from the partitionable shell, or until the partitionable shell is completely partitioned such that the tissue connecting elements become separated from each other.

According to some embodiments of the invention, each of the separated tissue connecting elements is provided beyond a different wall portion of the body tissue such that at least two consecutive separated tissue connecting elements are located at different sides of the body tissue opening.

According to some embodiments of the invention, the method further comprises: forcing the separated tissue connecting elements together against the stop element such that the separated tissue connecting elements converge along a final length of the thread while the body tissue opening substantially closes; and securing the separated tissue connecting elements to the final length of the thread.

According to some embodiments of the invention, forcing of the separated tissue connecting elements together results in an inverted suturing upon closing of the body tissue opening.

According to some embodiments of the invention, the method further comprises, between two of the repetitions: extending the thread to a selected extension length with a pulling force greater than the tensioning force; and locking the thread at the selected extension length.

According to some embodiments of the invention, the needle is provided in a shaft lumen of a shaft connected to an first jaw with a distal end thereof, the shaft lumen extending lengthwise therein and opened at the first jaw distal end; wherein the penetrating of the wall portion includes pushing the needle distally in the shaft lumen to a protruding position, in which the needle distal tip protrudes through the first jaw distal end at a maximal protrusion length; and wherein the retracting of the needle back from the wall portion includes pulling the needle proximally to a retracted position, in which the needle distal tip is inside the shaft lumen.

According to some embodiments of the invention, the second jaw is connected at distal end of the shaft or/and to the first jaw distal end configured to selectively actuate between a closed position and an opened position, the arm member includes a holder mechanism configured to arrest the distal-most tissue connecting element to the second jaw when the second jaw is at the closed position and when the distal-most tissue connecting element partially protrudes through the shaft distal end while the needle is in the retracted position.

According to some embodiments of the invention, retracting the needle includes applying the holder mechanism to arrest the distal-most tissue connecting element to the second jaw.

According to some embodiments of the invention, separating the distal-most tissue connecting element from the partitionable shell includes actuating the second jaw to shift from the closed position to the opened position.

According to some embodiments of the invention, the jaws are sized, shaped and arranged so as to grasp the body tissue having a thickness within a range of pre-determined allowed thickness values.

According to some embodiments of the invention, the method further comprises grasping the wall portion of the body tissue with the jaws.

According to some embodiments of the invention, separating the distal-most tissue connecting element from the partitionable shell includes releasing the wall portion from the grasping jaws.

According to some embodiments of the invention, the method further comprises activating the holder mechanism to release the distal-most tissue connecting element when the distal-most tissue connecting element extends completely beyond the internal surface of the wall portion.

According to an aspect of some embodiments of the present invention, there is provided a tissue connecting element, optionally having structure and function corresponding to a tissue suture anchor, comprising: a tubular wall extending between opposing ends, each of the opposing ends merges with a corresponding longitudinal opening, the longitudinal openings oppose each other and are separated with a strip, wherein each of the opposing ends of the tissue connecting element includes a contact surface having an area being at least half a cross section area of a tube having inner diameter and outer diameter same as of the tubular wall; wherein the tubular wall is configured to be strung and freely slide on a thread, and is sized and shaped to pass through a puncture in a body tissue wall when aligned towards the puncture and to revolve and press against the body tissue wall when pulled back via the thread; and wherein the tubular wall is further configured and arranged such that upon the thread being pulled back, the thread portion located therein shifts from extending between the opposing ends to wrapping around the strip.

According to some embodiments of the invention, a perimeter enclosing the contact surface comprises an outer part and an inner part, the perimeter outer part is greater than half a circumference of the tube.

According to some embodiments of the invention, the perimeter inner part includes two symmetrically opposing tangential lines touching both ends of a curve having an inner diameter same as the tube inner diameter.

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings;

FIGS. 1A-1D schematically illustrate side views of exemplary embodiments of a body tissue connecting device for closing an opening in body tissue, in accordance with some embodiments of the invention;

FIGS. 2A-2G schematically illustrate exemplary embodiments of possible scenarios in a method for closing an opening in body tissue, using an exemplary body tissue connecting device, in accordance some with embodiments of the invention;

FIGS. 3A-3G schematically illustrate different views of exemplary embodiments of a delivery device (and parts thereof) for delivering and deploying an exemplary body tissue connecting device, for closing an opening in body tissue, in accordance with some embodiments of the invention;

FIGS. 4A-4C schematically illustrate side cut views of exemplary embodiments of a needle track and a partly flexible needle for passing tissue connecting elements across a body tissue wall, and possible scenarios in a method for using thereof, in accordance with some embodiments of the invention;

FIGS. 5A-5C schematically illustrate different views of exemplary embodiments of a tissue connecting element, in accordance with some embodiments of the invention;

FIGS. 6A-6J schematically illustrate exemplary embodiments of a delivery device for delivering a body tissue connecting device, in accordance with some embodiments of the invention; and

FIGS. 7A-7S schematically illustrate exemplary embodiments of possible scenarios in a method for closing an opening in body tissue, in accordance with some embodiments of the invention.

### DESCRIPTION OF SPECIFIC EMBODIMENTS OF THE INVENTION

The present invention, in some embodiments thereof, relates to devices and methods for closing openings in body tissue. Some embodiments of the present invention particularly relate to a delivery device for delivering a body tissue connecting device; a body tissue connecting device; methods using thereof; tissue connecting elements (for example, as a form of tissue suture anchors); and applications thereof, such as for closing openings in body tissue, for example, associated with cuts, incisions, or other soft tissue discontinuities.

The following exemplary embodiments may be described in the context of suture anchors for amending or otherwise affecting body soft tissues. However, the invention is not limited to the specifically described devices and methods, and may be adapted to various clinical applications without departing from the overall scope of the invention.

The present invention, in some embodiments thereof, may be implemented with tissue connecting elements having structural and functional characteristics and features of suture anchors. The term “suture anchor”, as used herein, refers to any surgical artifact that is configured for mechanical connectivity with a body tissue, optionally a soft tissue, and interconnects with other similar anchors with a thread, such as a suture. In exemplary embodiments, the tissue connecting elements in a form of suture anchors are designed and configured as “T anchors”, whereby, upon deployment thereof with a thread interlinking therebetween, an anchor will be pressed against a tissue portion by pulling the thread towards and through the tissue portion, whereas the thread (either one end or two ends thereof) passes through the anchor via opening at or adjacent a mid-length thereof.

Some embodiments of the invention are characterized by the following exemplary features, which may be beneficial compared to prior art techniques of closing openings in body tissue. During implementation of some embodiments, the thread (suture) is substantially tensioned throughout the delivery, as well as at steps (procedures) of delivering the tissue connecting elements (anchors) and attaching the tissue connecting elements (anchors) to the body tissue. Optionally and additionally, during delivery of tissue connecting elements (anchors) and thread (suture), the tissue connecting elements (anchors) are compacted one to the other so that the thread (suture) may be covered with the tissue connecting elements (anchors), optionally though not necessarily with minimal or no gaps therebetween, or/and with no overlapping or non-coinciding between each two adjacent tissue connecting elements (anchors), in an effort to avoid possible entangling of the thread (suture) with the tissue connecting elements (anchors). In some such embodiments, some or all tissue connecting elements (anchors) are compressed distally against a stop element, such as, illustratively, beads threaded together on a thread (string) and compressed against a bead-type stop element (bead-stop) permanently fixated to the thread (string) distal end. In exemplary embodiments, the thread does not extend much or at all distally to the stop is element.

An aspect of some embodiments of the present invention relates to a body tissue connecting device for closing an opening in body tissue. In exemplary embodiments, the body tissue connecting device includes a thread fixedly connected at a distal end thereof to a stop element, and a plurality of tissue connecting elements (optionally at least three, optionally at least nine tissue connecting elements) strung on the thread proximally to the stop element. The tissue connecting elements are compactable to a selected configuration along the thread upon compression thereof between the stop element and a support element (optionally provided proximally to the tissue connecting elements). In some embodiments, the tissue connecting elements are functional as suture anchors, optionally visceral anchors, for treating or otherwise affecting tissues, such as soft tissues. In some embodiments, each of the tissue connecting elements is sized and shaped to pass through a puncture in a body tissue wall when aligned towards the puncture and to revolve and press against the body tissue wall when pulled back with the thread.

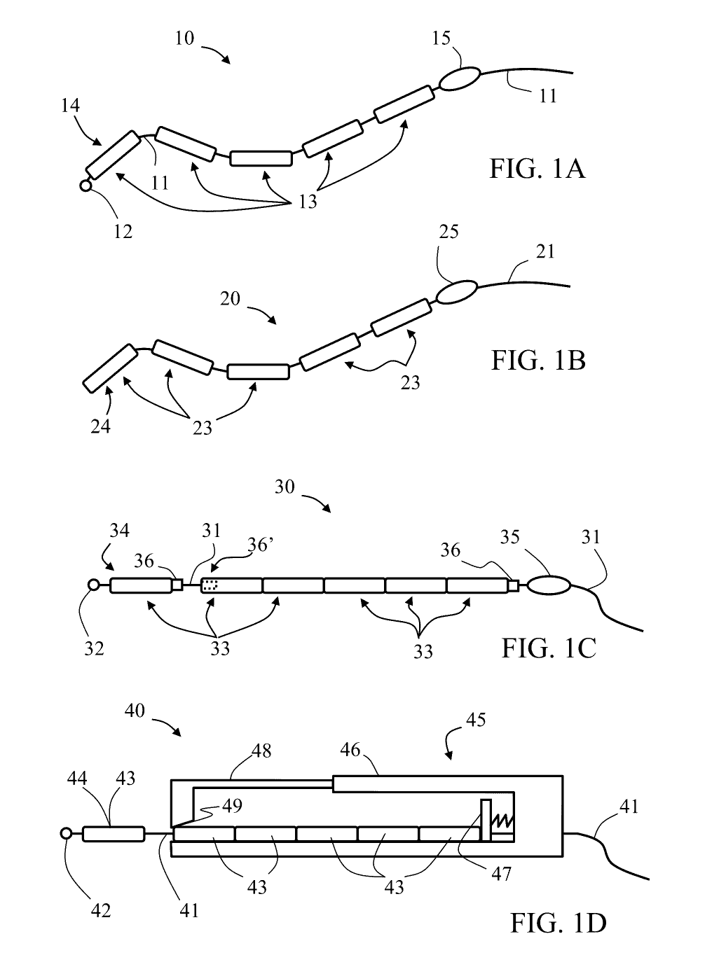

Referring now to the drawings, FIGS. 1A-D schematically illustrate side views of exemplary embodiments of a body tissue connecting device for closing an opening in body tissue (herein, also referred to as a body opening (BO)). FIG. 1A shows a body tissue connecting device 10 that includes a thread 11 and is fixedly connected at a distal end thereof to a stop element 12. Thread 11 may be a biocompatible or/and implant grade thread such as a suture. A plurality of elongated tissue connecting elements 13 is strung on thread 11, proximally to stop element 12, functional as separate suture anchors (e.g., in a form of “T-anchors” as commonly referred to in relevant art). The figure shows five tissue connecting elements although there can be any number of tissue connecting elements, optionally at least three, optionally at least nine, optionally at least 15, or higher, or lower, or an intermediate number. In some embodiments, tissue connecting elements 13 are provided in the form of a partitionable (separable) shell, meaning that they provide a continuous jacket to thread 11 passing therethrough. Optionally, the partitionable (separable) shell is in a form of a closed tube so that the tissue connecting elements are gaplessly compacted one with the other, and each tissue connecting element does not include openings at its periphery, at least not openings which are large enough for thread 11 to pass through or/and entangle with.

In some embodiments, at least one of tissue connecting elements 13 is not in a form of a completely close tube. In some such embodiments, compacting two adjacent tissue connecting elements 13, by adjoining a contact surface in adjacent opposing side of each tissue connecting element 13, will result in forming a gap therebetween, along portion of the circumference around each contact surface. Maximizing the contact surface areas of adjacent tissue connecting elements 13 optionally corresponds to maximal alignment of the lumenal areas of the adjacent tissue connecting elements 13. Optionally, in order to avoid non-coincidence of each two adjacent tissue connecting elements 13, when compacted one against the other, the area of each contact surface is at least half the cross section area of a tube having inner diameter and outer diameter same as of said tubular wall.

Tissue connecting elements 13 are unconnected with each other and can freely slide over thread 11 distally (i.e., towards stop element 12) or proximally, such as beads threaded together on a string. Tissue connecting elements 13 are optionally elastic, semi-elastic or/and bendable, optionally in order to pass through non-linear (e.g., curved) channels during delivery. Tissue connecting elements 13 include a distal-most tissue connecting element 14 capable of being in direct contact with stop element 12. Stop element 12 may be sized, which may be shaped and configured such as a blocking member, has a diameter greater than an inner diameter of distal-most component 14, so that to prevent movement of distal-most tissue connecting element 14 distally therethrough. Instead or in addition to a stop element in a form of a blocking member, thread 11 may be provided with a knot at a distal portion thereof, optionally in close proximity to its distal free end, or that such a knot may be knotted in preparation to the medical procedure.

A support element 15 is provided strung on thread 11 proximally to all tissue connecting elements 13. Optionally, support element 15 can be selectively fixated at any selected point along thread 11 proximally to tissue connecting elements 13. Instead of support 15, other means can be used to secure a selected length of thread 11, such as a knot (e.g., slip knot). Optionally and additionally, support element 15 can be used as a pusher member being selectively applicable to apply a compressive force to elongated tissue connecting elements 13. Tissue connecting elements 13 are therefore compactable to a selected configuration along thread 11 upon compression thereof between stop element 12 and support element 15; optionally a selected configuration is achieved by using a shaped channel (not shown) for housing the tissue connecting elements compacted together and assuming the channel shape, Optionally, support element 15 can be used as a locking member applicable to lock elongated tissue connecting elements 13 along a selected length of thread 11 between support element 15 and stop element 12.

FIG. 1B shows a body tissue connecting device 20 mostly similar in components and function to body tissue connecting device 10 despite excluding a stop element differentiated at least in size or/and shape than the elongated tissue connecting elements. Body tissue connecting device 20 includes a thread 21 and is fixedly connected at a distal end thereof to a distal-most tissue connecting element 24. The stop element is then a distal-most component of a plurality of tissue connecting elements. Distal-most tissue connecting element 24 is therefore configured to function also as a stop element for other elongated tissue connecting elements 23 being strung on thread 21 proximally thereto and functional as separate tissue anchors. Thread 21 may be a biocompatible or/and implant graded thread such as a suture. The figure shows five tissue connecting elements although there can be any number of tissue connecting elements, optionally at least three, optionally at least nine, optionally at least 15, or higher, or lower, or an intermediate number. In some embodiments, tissue connecting elements 23 are provided in the form of a partionable (separable) shell, meaning that they provide a continuous gapless jacket to thread 21 passing therethrough. Optionally, the partitionable (separable) shell is in a form of a closed tube so that the tissue connecting elements are gaplessly compacted one with the other, and each tissue connecting element does not include openings at its periphery, at least not openings which are large enough for thread 21 to pass through or/and entangle with.

In some embodiments, at least one of tissue connecting elements 23 is not in a form of a completely close tube. In some such embodiments, compacting two adjacent tissue connecting elements 23, by adjoining a contact surface in adjacent opposing side of each tissue connecting element 23, will result in forming a gap therebetween, along portion of the circumference around each contact surface. Maximizing the contact surface areas of adjacent tissue connecting elements 23 optionally corresponds to maximal alignment of the lumenal areas of the adjacent tissue connecting elements 23. Optionally, in order to avoid non-coincidence of each two adjacent tissue connecting elements 23, when compacted one against the other, the area of each contact surface is at least half the cross section area of a tube having inner diameter and outer diameter same as of said tubular wall.

Tissue connecting elements 23 are unconnected with each other and, besides distal-most tissue connecting element 24, can freely slide over thread 21 distally or proximally, such as beads threaded together on a string. Tissue connecting elements 23 are optionally elastic, semi-elastic or/and bendable, optionally in order to pass through non-linear (e.g., curved) channels during delivery.

A support element 25 is provided strung on thread 21 proximally to all tissue connecting elements 23. Optionally, support element 25 can be selectively fixated at any selected point along thread 21 proximally to tissue connecting elements 23. Instead of support element 25, other means can be used to secure a selected length of thread 21, such as a knot (e.g., slippery knot). Optionally and additionally, support element 25 can be used as a pusher member being selectively applicable to apply a compressive force to elongated tissue connecting elements 23. Tissue connecting elements 23 are therefore compactable to a selected configuration along thread 21 upon compression thereof between distal-most tissue connecting element 24 and support element 25; optionally a selected configuration is achieved by using a shaped channel (not shown) for housing the tissue connecting elements compacted together and assuming the channel shape. Optionally, support element 25 can be used as a locking member applicable to lock elongated tissue connecting elements 23 along a selected length of thread 21 between support element 25 and distal-most tissue connecting element 24.

FIG. 1C shows a body tissue connecting device 30 mostly similar in components and function to apparatus 10 despite having the elongated tissue connecting elements releasably connectable with each other. Body tissue connecting device 30 includes a thread 31 and is fixedly connected at a distal end thereof to a stop element 32. Thread 31 may be a biocompatible or/and implant graded thread such as a suture. A plurality of elongated tissue connecting elements 33 is strung on thread 31, proximally to stop element 32, functional as separate suture anchors. The figure shows six tissue connecting elements although there can be any number of tissue connecting elements, optionally at least three, optionally at least nine, optionally at least 15, or higher, or lower, or an intermediate number. In some embodiments, tissue connecting elements 33 are provided in the form of a partitionable (separable) shell, meaning that they provide a continuous gapless jacket to thread 31 passing therethrough. Optionally, the partitionable (separable) shell is in a form of a closed tube so that the tissue connecting elements are gaplessly compacted one with the other, and each tissue connecting element does not include openings at its periphery, at least not openings which are large enough for thread 31 to pass through or/and entangle with.

In some embodiments, at least one of tissue connecting elements 33 is not in a form of a completely close tube. In some such embodiments, compacting two adjacent tissue connecting elements 33, by adjoining a contact surface in adjacent opposing side of each tissue connecting element 33, will result in forming a gap therebetween, along portion of the circumference around each contact surface. Maximizing the contact surface areas of adjacent tissue connecting elements 33 optionally corresponds to maximal alignment of the lumenal areas of the adjacent tissue connecting elements 33. Optionally, in order to avoid non-coincidence of each two adjacent tissue connecting elements 33, when compacted one against the other; the area of each contact surface is at least half the cross section area of a tube having inner diameter and outer diameter same as of said tubular wall.

Tissue connecting elements 33 are releaseably connected with each other; and optionally each tissue connecting element 33 includes a male connector 36 snugly fitting in a female connector 36′ provided in an opposing end of an adjacent tissue connecting element. Tissue connecting elements 33 can freely slide over thread 31 distally (i.e., towards stop element 32) or proximally, such as beads threaded together on a string, either as a single member (if interconnect) or separately. Tissue connecting elements 33 are optionally elastic, semi-elastic or/and bendable, optionally in order to pass through non-linear (e.g., curved) channels during delivery. Optionally, the connectors of the tissue connecting elements allow degree of freedom for each two interconnected tissue connecting elements. Tissue connecting elements 33 include a distal-most tissue connecting element 34 capable of being in direct contact with stop element 32. Stop element 32, which may be sized, shaped and configured such as a blocking member, has a diameter greater than an inner diameter of distal-most component 34, so that to prevent movement of distal-most tissue connecting element 34 distally therethrough. Instead or in addition to a stop element in a form of a blocking member, thread 31 may be provided with a knot at a distal portion thereof, optionally in close proximity to its distal free end, or that such a knot may be knotted in preparation to the medical procedure.

A support element 35 is provided strung on thread 31 proximally to all tissue connecting elements 33. Optionally, support element 35 can be selectively fixated at any selected point along thread 31 proximally to tissue connecting elements 33. Instead of support element 35, other means can be used to secure a selected length of thread 31, such as a knot (e.g., slippery knot). Optionally and additionally, support element 35 can be used as a pusher member being selectively applicable to apply a compressive force to elongated tissue connecting elements 33. Tissue connecting elements 33 are therefore compactable to a selected configuration along thread 31 upon compression thereof between stop element 32 and support element 35; optionally a selected configuration is achieved by using a shaped channel (not shown) for housing the tissue connecting elements compacted together and assuming the channel shape. Optionally, support element 35 can be used as a locking member applicable to lock elongated tissue connecting elements 33 along a selected length of thread 31 between support element 35 and stop element 32.

FIG. 1D shows a body tissue connecting device 40 mostly similar in components and function to body tissue connecting device 10 provided in a dispensing assembly 45. Body tissue connecting device 40 includes a thread 41 and is fixedly connected at a distal end thereof to a stop element 42. Thread 41 may be a biocompatible or/and implant graded thread such as a suture. A plurality of elongated tissue connecting elements 43 is strung on thread 41, proximally to stop element 42, functional as separate suture anchors. The figure shows six tissue connecting elements although there can be any number of tissue connecting elements, optionally at least three, optionally at least nine, optionally at least 15, or higher, or lower, or an intermediate number. In some embodiments, tissue connecting elements 43 are provided in the form of a partitionable (separable) shell, meaning that they provide a continuous gapless jacket to thread 41 passing therethrough. Optionally, the partitionable (separable) shell is in a form of a closed tube or a non-closed tube and the tissue connecting elements are compactable.

Tissue connecting elements 43 are unconnected with each other and can slide over thread 41 distally (i.e., towards stop element 42) or proximally, such as beads threaded together on a string, either as a single member (if interconnect) or separately. Tissue connecting elements 43 are optionally elastic, semi-elastic or/and bendable, optionally in order to pass through non-linear (e.g., curved) channels during delivery. Tissue connecting elements 43 include a distal-most tissue connecting element 44 capable of being in direct contact with stop element 42. Stop element 42, which may be sized, shaped and configured such as a blocking member, has a diameter greater than an inner diameter of distal-most component 44, so that to prevent movement of distal-most tissue connecting element 44 distally therethrough. Instead or in addition to a stop element in a form of a blocking member, thread 41 may be provided with a knot at a distal portion thereof, optionally in close proximity to its distal free end, or that such a knot may be knotted in preparation to the medical procedure. In some embodiments, each pair of adjacent tissue connecting elements 43, when gaplessly compacted to a partionable (separable) shell, includes a first rotationally asymmetric tissue connecting element identical in shape and size to a second tissue connecting element. Optionally, the first tissue connecting element is rotated, optionally in a straight angle, relatively to the second tissue connecting element, around a shared longitudinal axis thereof.

Dispensing assembly 45 may also function as a support member for supporting or/and compacting the tissue connecting elements, such as described above and shown in FIGS. 1A, 1B, and 1C. Dispensing assembly 45 comprises a housing 46 with a supporting surface 47, optionally spring loaded (as shown) for supporting tissue connecting elements 43 against either stop element 42 or a restraint 49. Instead of dispensing assembly 45, other means can be used to secure a selected length of thread 41, such as a knot (e.g., slippery knot). Dispensing assembly 45 may be used to compact all or some tissue connecting elements 43 together, such as all tissue connecting elements besides distal-most tissue connecting element 44 (as shown) so that while distal-most tissue connecting element 44 may be anchored to a tissue portion, the other tissue connecting elements 43 are kept compacted (between supporting surface 47 and restraint 49) with minimal gaps therebetween, at least until a trailing tissue connecting element (a “new” distal-most tissue connecting element) is pushed/pulled out from housing 46 and used.

Restraint 49 may be length-fixed with an arm 48 extending from housing 46, or may extend or retract with respect to housing 46 as needed, and may optionally include a length-changeable (e.g., telescopic) arm 48. Restraint 49 or/and arm 48 may be resistive to shape deformation and have elastic characteristics such that upon pushing or pulling distally the tissue connecting element 43 above a pre-determined magnitude, restraint 49 will cease to hold at least one tissue connecting element from moving distally. Dispensing assembly 45 is provided strung on thread 41 proximally to all tissue connecting elements 43. Optionally, dispensing assembly 45 can be selectively fixated at any selected point along thread 41 proximally to tissue connecting elements 43. Optionally and additionally, dispensing assembly 45 can be used as a pusher member being selectively applicable to apply a compressive force to elongated tissue connecting elements 43.

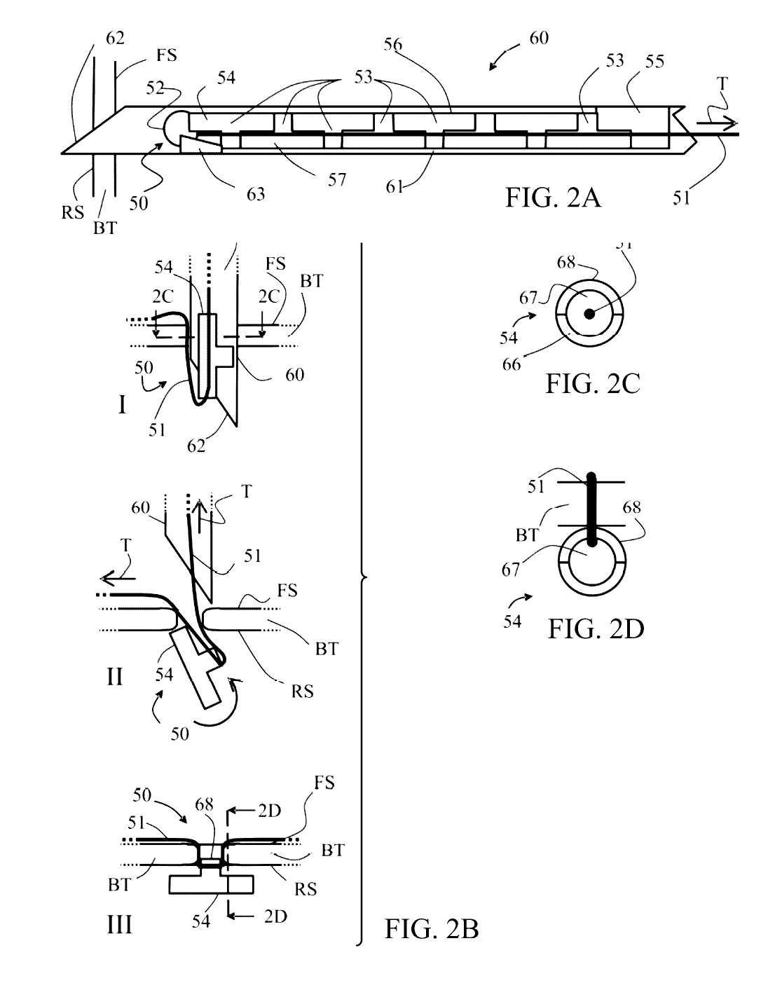

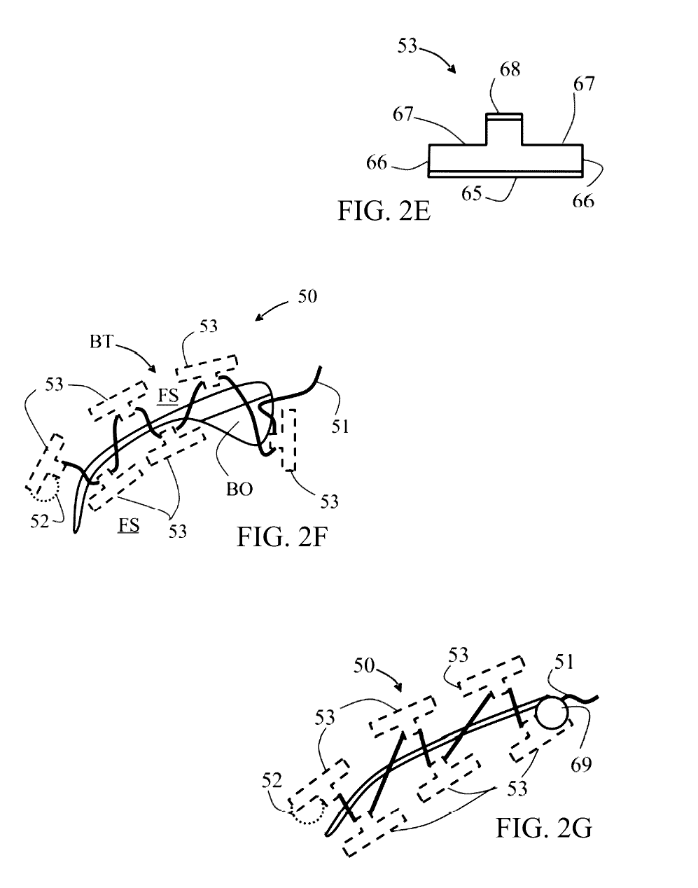

FIGS. 2A-2G schematically illustrate exemplary embodiments of possible scenarios in a method for closing an opening in body tissue using an exemplary body tissue connecting device 50. Body tissue connecting device 50 includes a thread 51 and is fixedly connected at a distal end thereof to a stop element 52. Thread 51 may be a biocompatible or/and implant grade thread such as a suture. A plurality of elongated tissue connecting elements 53 is strung on thread 51, proximally to stop element 52, functional as separate suture anchors for deployment in a body. The figure shows eight tissue connecting elements although there can be any number of tissue connecting elements, optionally at least three, optionally at least nine, optionally at least 15, or higher, or lower, or an intermediate number. FIG. 2A shows a side cut view of body tissue connecting device 50 during delivery phase, tissue connecting elements 53 are provided and delivered (e.g., prior to deployment) in the form of a partitionable (separable) shell 56, and configured to provide a continuous jacket to thread 51 passing therethrough. Optionally, the partitionable shell is in a form of a closed tube so that the tissue connecting elements are gaplessly compacted one with the other, and each tissue connecting element does not include openings at its periphery, at least not openings which are large enough for thread 51 to pass through or/and entangle with.

Tissue connecting elements 53 are unconnected with each other and can freely slide over thread 51 distally (i.e., towards stop element 52) or proximally, such as beads threaded together on a string. Tissue connecting elements 53 are optionally elastic, semi-elastic or/and bendable, or alternatively substantially rigid. Tissue connecting elements 53 include a distal-most tissue connecting element 54 capable of being in direct contact with stop element 52. Stop element 52, is sized, shaped and configured such as a blocking member having a diameter greater than an inner diameter of distal-most component 54, so that to prevent movement of distal-most tissue connecting element 54 distally therethrough and relative thereto. Instead or in addition to a stop element in a form of a blocking member, thread 51 may be provided with a knot at a distal portion thereof, optionally in close proximity to its distal free end, or that such a knot may be knotted in preparation to the medical procedure.

A delivery support 55 is provided strung on thread 51 proximally to all tissue connecting elements 53. Optionally, delivery support 55 is a pusher member selectively applicable to apply a compressive force to elongated tissue connecting elements 53. Tissue connecting elements 53 are compactable to a selected configuration along thread 51 upon compression thereof between stop element 52 and delivery support 55 and within the boundaries imposed by a lumen of a delivery device.

In some embodiments, body tissue connecting device 50 is delivered to a target location for closing an opening in a body tissue BT using a device with a needle 60 which comprises a needle lumen 61 and a needle distal tip 62. Needle distal tip 62 is pointed to facilitate penetration through the target body tissue BT. Needle lumen 61 is sized to accommodate tissue connecting elements 53 strung on thread 51. A restraint 63 is optionally provided in needle lumen 61 adjacent needle distal tip 62 for restraining tissue connecting element 53, optionally distal-most tissue connecting element 54, from passing distally therethrough—up to a maximal restraining force. As shown, tissue connecting elements 53 are compacted to a partitionable (separable) shell configuration 56 jacketing thread 51 therein, when pressed against restraint 63 with a pushing force equal to or smaller than the maximal restraining force. In some embodiments, restraint 63 is located at a particular location along the length of needle 60 in lumen 61 such that upon partial protrusion of distal-most tissue connecting element 54 through the needle distal tip 62, restraint 63 shall restrain a trailing tissue connecting element 57 immediately following distal-most tissue connecting element 54. Throughout the delivery phase shown in the scenario of FIG. 2A, thread 51 is optionally tensioned with a tensioning force T (optionally, constant force, or optionally, within a predetermined range of forces) in order to avoid being tangled. Thread 51 tensioning is optionally between stop element 52 and a point at the thread proximally to partitionable (separable) shell 56. A wall portion of body tissue BT, adjacent a body opening BO (FIG. 2F), can be penetrated with needle distal tip 62 such that needle distal tip 62 moves from pointing towards an external (frontal) surface FS of the wall portion to protruding beyond an internal (rear) surface RS of the wall portion.

FIG. 2B shows partial side cut views of body tissue connecting device 50 at three exemplary consecutive scenarios I, II and III (possibly with separations therebetween) of delivering and deploying distal-most tissue connecting element 54 to connect with body tissue BT following a full penetration therethrough of needle 60 such that needle lumen 61 is exposed beyond body tissue BT in full diameter. As shown, distal-most tissue connecting element 54 may not necessarily be a first tissue connecting element to be delivered but rather most distal tissue connecting element still maintained fully or partially within needle lumen 61.

In scenario I—needle 60 is shown fully penetrated through body tissue BT from a first side to an opposing side thereof, with needle distal tip 62. Distal-most tissue connecting element 54 is passed from needle lumen 61 and emerges beyond internal surface RS, optionally being pulled or pushed, or drops, therein. Thread 51 extends generally lengthwise across distal-most tissue connecting element 54 and between proximal to distal sides thereof, then passes out of needle lumen 61 and back (in a proximal direction) through the perforation of body tissue BT along outer wall of needle 60. Optionally, alternatively or additionally, needle 60 is retracting from the wall portion while distal-most tissue connecting element 54 is kept (e.g., held) beyond internal surface RS of the wall portion such that needle 60 exposes distal-most tissue connecting element 54. Distal-most tissue connecting element 54 is separated and spaced from partitionable (separable) shell 56, optionally before, during or after occurrence of scenario I. Thread 51 is optionally not tensioned when there is a need for maneuverability of distal-most tissue connecting element 54.

In some embodiments, each tissue connecting element 53 is sized and shaped to pass through a puncture in a body tissue wall when aligned towards the puncture and to revolve and press against the body tissue wall when pulled back with thread 51. In scenario II of FIG. 2B needle 60 is retracted from body tissue BT and thread 51 is pulled proximally, optionally though not necessarily with substantially same tensioning force T, forcing distal-most tissue connecting element 54 to revolve and eventually press against the body tissue wall at the internal surface RS thereof.

In some embodiments, and as shown in greater details in FIGS. 2C-2E, each tissue connecting element 53 includes a tubular wall 65 extending between opposing ends 66, each opposing end 66 merges with a corresponding longitudinal opening 67, the longitudinal openings 67 opposes each other and separated with a strip 68. As such, tissue connecting element 53 is configured and arranged such that upon thread 51 being pulled back (e.g., proximally) the thread portion strung therein shifts from extending between opposing ends 66 (as shown in FIG. 2C) to wrapping around strip 68 (as shown in FIG. 2D), Optionally, the edges surrounding each of longitudinal openings 67 are outlined such to allow the thread portion strung therein to unhinderedly slip thereon or/and pass above towards strip 68.

Scenario III in FIG. 2B shows distal-most tissue connecting element 54 in a fully deployed position being pressed against internal surface RS while thread 51 wraps around strip 68, travels in both portions extending out of strip 68 through the (optionally healing) perforation in body tissue BS, and pass towards other proximate tissue connecting elements (deployed or delivered).

In some embodiments, each pair of adjacent tissue connecting elements 53, when gaplessly compacted to a partitionable (separable) shell, includes a first rotationally asymmetric tissue connecting element identical in shape and size to a second tissue connecting element. Optionally, the first tissue connecting element is rotated, optionally in a straight angle, relatively to the second tissue connecting element, around a shared longitudinal axis thereof.

In some embodiments, the above mentioned steps are repeated, each time with a “new” distal-most tissue connecting element consecutive to a “previous” distal-most tissue connecting element, until a selected number of tissue connecting elements 53 is separated from partitionable (separable) shell 56 or until partitionable (separable) shell 56 is completely partitioned (separated) into separated tissue connecting elements 53.

FIG. 2F schematically illustrates a top view of body tissue connecting device 50 in midst of deployment around body opening BO in body tissue BT, where six tissue connecting elements 53 have already been placed beyond external surface FT (therefore the deployed tissue connecting elements 53 as well as stop element 52 are shown in dashed lines). Each separated tissue connecting element 53 is provided beyond a different body tissue wall portion such that each two consecutive separated tissue connecting elements 53 are located at different sides of the body opening BO. Thread 51 extends from stop element 52 through all six tissue connecting elements 53, wrapping around the strips of the tissue connecting elements and otherwise maintained above body tissue BT and external surface FS.

As shown, body opening BO is partly closed due to the repetitive tensioning of thread 51 after each local deployment of a tissue connecting element and activation thereof as a suture anchor. This is considered unique relative to other suture anchor devices and methods of use thereof which are incapable of or/and not applied to close body openings while deploying all anchors to the tissue, but only after all anchors are in place.

FIG. 2G illustrates a top view of body tissue connecting device 50 after completion of deployment where body opening is closed and secured to a selected length of thread 51. In shifting from the scenario shown in FIG. 2F to the scenario of FIG. 2G, thread 51 was pulled, optionally using a pulling force substantially greater than tensioning force T, and a support element 69 was placed proximally to tissue connecting elements 53 and used as a locking member to lock elongated tissue connecting elements 53 along a selected length of thread 51 between support 69 and stop element 52.

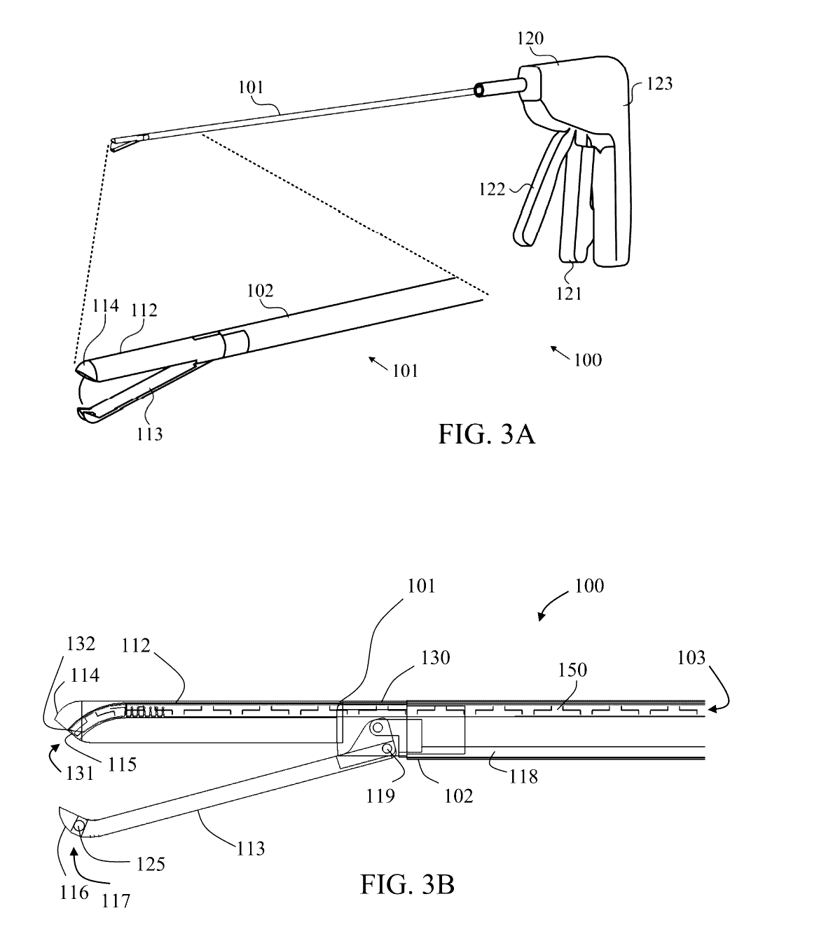

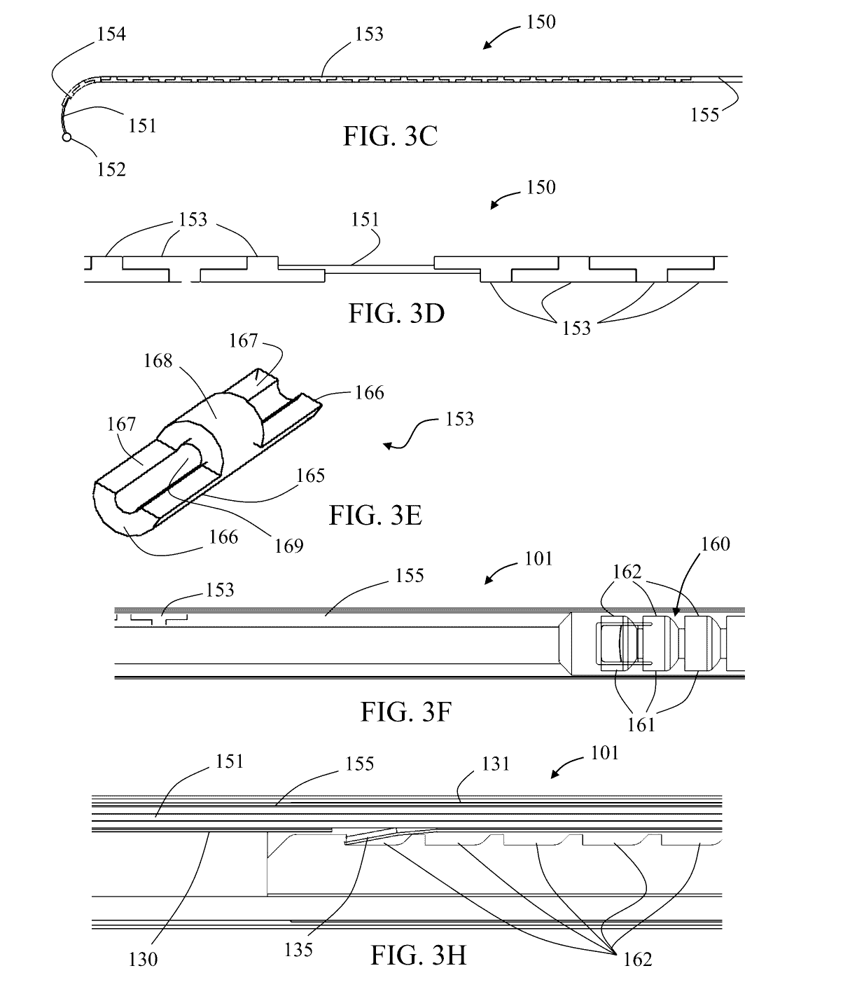

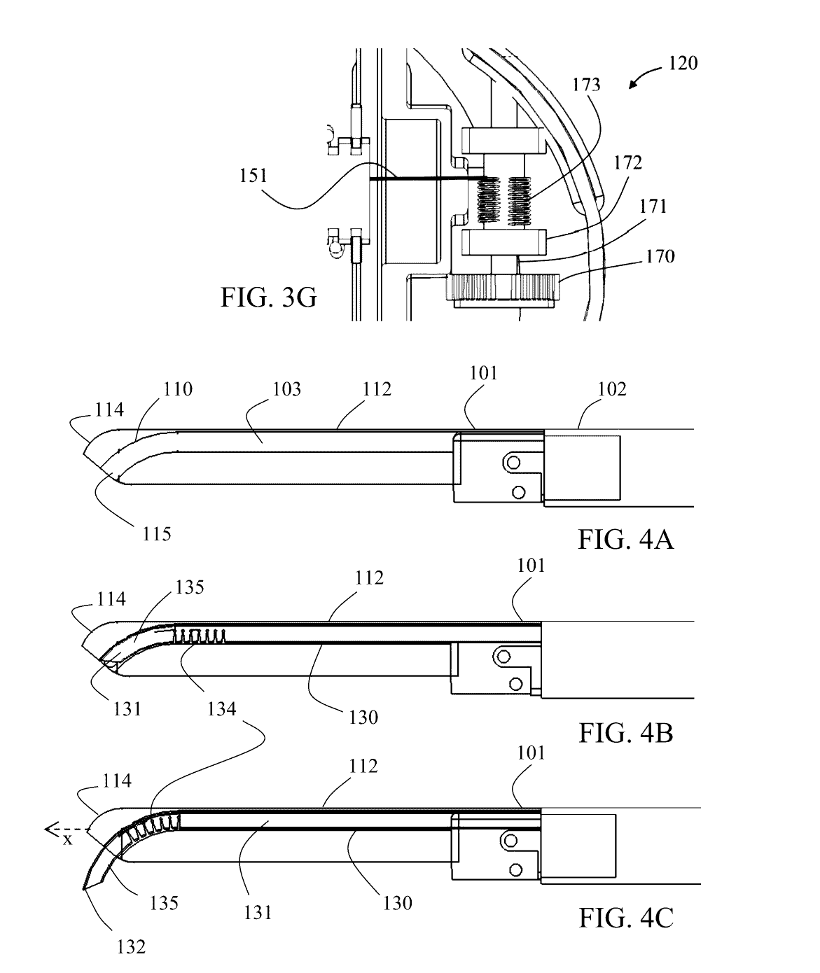

Reference is now made to FIGS. 3A-3G, which schematically illustrate different views of exemplary embodiments of a delivery device 100, and parts thereof, applicable for delivering and deploying an exemplary body tissue connecting device 150, for closing an opening in body tissue. In some embodiments, delivery device 100 is sized and configured for laparoscopic or endoscopic surgeries. In some such embodiments, at delivery mode into the body and towards the target body tissue, device 100 has a maximal outer diameter equal to or less than 12 mm, optionally equal to or less than 5 mm, optionally equal to or less than 3 mm, or higher, or lower, or an intermediate diameter. As shown in FIG. 3A, delivery device 100 includes a shaft 101 connected at its proximal end to a handle assembly 120. Handle assembly 120 comprises at least one actuator, optionally an arm actuator 121 and a needle actuator 122. Device 100 comprises a pair of opposing jaws: a first, optionally static, jaw 112 and a second, movable, jaw 113. Shaft 101 is connected at a distal end 102 thereof to first jaw 112, and both are sharing a shaft lumen 103 (shown in FIG. 3B) extending lengthwise therein and opened at distal end 114 of first jaw 112. Second jaw 113 is pivotally connected to distal end 102 of shaft 101 (or optionally to first jaw 112). The jaws are sized, shaped and arranged such to grasp a body tissue with a thickness within a range of pre-determined allowed thickness values. An exemplary range of pre-determined allowed thickness values is between about 0.1 mm and about 10 mm.

As shown in FIG. 3B, shaft lumen 103 is opened at an opening 115 in first jaw distal end 114. A needle 130 extends in shaft lumen 103 and includes a needle lumen 131 and a needle distal tip 132. In some embodiments, needle lumen 131 is sized to accommodate apparatus 150 which comprises a plurality of tissue connecting elements 153 (for ease of explanation only, needle 130 is illustrated in FIG. 3B as transparent hence showing tissue connecting elements 153). In some embodiments, the jaws are required especially in case of unnaturally rigidly supported soft tissues, such as visceral organs tissues, so that one of the jaws can provide counter rigid support around the penetration area/point formed by needle distal tip 132. In some embodiments, a distal end 116 of second jaw 113 is angled (e.g., bent) towards distal end 114 of first jaw 112. Second jaw 113 is pivotally connected to shaft distal end 102 with a pivot connection 119. Second jaw 113 includes a jaw opening 117 sized to allow needle distal tip 132 passing therethrough, wherein upon grasping a body tissue, needle distal tip 132, when protruded to the maximal protrusion length, passes through jaw opening 117 and penetrating through the body tissue. A jaw actuator 121 is interconnected with second jaw 113 using a sliding bar 118, and can be used selectively to actuate second jaw 113 between a closed position and an opened position (shown in FIG. 3B).

FIG. 3C shows body tissue connecting device 150 in an exemplary delivery configuration, as in delivery device 100, prior to deployment of any of tissue connecting elements 153. FIG. 3D is a close-up view of an intermediate portion of apparatus 150 presenting spaced apart tissue connecting elements 153 for demonstrative purpose only for showing a thread 151 passing therethrough. Thread 151 is fixedly connected at a distal end thereof to a stop element 152. Thread 151 may be a biocompatible or/and implant graded thread such as a suture. The plurality of elongated tissue connecting elements 153 is strung on thread 151, proximally to stop element 152, functional as separate suture anchors. As shown, tissue connecting elements 153 are provided and delivered (e.g., prior to deployment) in the form of a partitionable (separable) shell, meaning that they provide a continuous jacket to thread 151 passing therethrough. Optionally, the partitionable (separable) shell is in a form of a closed tube so that the tissue connecting elements are gaplessly compacted one with the other, and each tissue connecting element does not include openings at its periphery, at least not openings which are large enough for thread 151 to pass through or/and entangle with. Tissue connecting elements 153 are unconnected with each other and can freely slide over thread 151 distally (i.e., towards stop element 152) or proximally, such as beads threaded together on a string. Tissue connecting elements 153 are optionally elastic, semi-elastic or/and bendable. Optionally, tissue connecting elements 153 are made of medical or implant grade stainless steel or polymeric material such as PEEK.

Tissue connecting elements 153 include a distal-most tissue connecting element 154 capable of being in direct contact with stop element 152. Stop element 152 is sized, shaped and configured such as a blocking member having a diameter greater than an inner diameter of distal-most component 154, so that to prevent movement of distal-most tissue connecting element 154 distally therethrough, Instead or in addition to a stop element in a form of a blocking member, thread 151 may be provided with a knot at a distal portion thereof, optionally in close proximity to its distal free end, or that such a knot may be knotted in preparation to the medical procedure. In some embodiments, stop element is at least 0.1 mm in diameter, optionally equal to or greater than 0.3 mm, optionally equal to or greater than 0.5 mm. In some embodiments, each tissue connecting element 153 and stop element 152 is equal to or smaller than in outer diameter to inner diameter of needle lumen 131, or is conformable (e.g., compressible) is shaped or/and size to needle lumen 131 inner boundaries. In some embodiments, tissue connecting elements 153 and stop element 152 are equal to or smaller than or conformable to an outer dimension of 3 mm, optionally 1.5 mm, optionally 0.9 mm, optionally 0.5 mm.

A delivery support 155 is provided strung on thread 151 proximally to all tissue connecting elements 153, functional as a pusher member selectively applicable to apply a compressive force to elongated tissue connecting elements 153. Tissue connecting elements 153 are compactable to a selected configuration along thread 151 upon compression thereof between stop element 152 and delivery support 155 and within the boundaries imposed by shaft lumen 103 of a device 100. In some embodiments, each pair of adjacent tissue connecting elements 153, when gaplessly compacted to a partionable (separable) shell, includes a first rotationally asymmetric tissue connecting element identical in shape and size to a second tissue connecting element. Optionally, the first tissue connecting element is rotated, optionally in a straight angle, relatively to the second tissue connecting element, around a shared longitudinal axis thereof.

In some embodiments, and as shown in greater details in FIG. 3E, each tissue connecting element 153 includes a tubular wall 165 extending between opposing ends 166, each opposing end 166 merges with a corresponding longitudinal opening 167, the longitudinal openings 167 opposes each other and separated with a strip 168. A lumen 169 passes through strip 168 and opens to two opposing channels corresponding to the two longitudinal openings 167. A plurality of tissue connecting elements 153 may be manufactured by cutting a single tube using a series of spaced mirrored “S” cuts. As such, each tissue connecting element 153 is configured and arranged such that upon thread 151 being pulled back (e.g., proximally) the thread portion strung therein shifts from extending between opposing ends 166 to wrapping around strip 168. Optionally, the edges surrounding each of longitudinal openings 167 are outlined such to allow the thread portion strung therein to unhinderedly slip thereon or/and pass above towards strip 168.

FIGS. 3F-3H show side cut views of different magnified portions of shaft 101. Delivery support or pusher 155 is provided in needle lumen 131 and is hollow such to allow thread 151 passing lengthwise therethrough unhinderedly. Pusher 155 comprises or is fixedly connected to a linear ratchet 160 comprising a first line of spaced indentations 161, each indentation corresponds to a tissue connecting element 153 in length, such that pusher 155 is restricted from proximal progression and each sequenced distal progression thereof is configured to push a first tissue connecting element 153 to an area in shaft lumen 103 previously occupied by a distally adjacent tissue connecting element 153 of same length. In some embodiments, a tooth (not shown) fixated to shaft 101 is projected inwardly within one of indentations 161 and shaped and directed such that linear ratchet 160 can progress distally and restricted from progressing proximally.

Linear ratchet 160 comprises a second line of spaced indentations 162, each indentation corresponds to the maximal protrusion length of needle 130 via opening 115 in shaft distal end 114. Needle 130 can slide between the protruding towards position and the retracted position notwithstanding pusher 155 position in shaft lumen 103. Needle 130 includes a tooth 135 protruding towards linear ratchet 160 so in case it is positioned in a particular indentation 162 it permits relative motion with respect to linear ratchet 160 within the boundaries of this particular 162 or to a proximally adjacent indentation but not distally to the particular distention 162. Needle 130 is linked with needle actuator 122 such that upon pressing needle actuator 122 needle 130 is pushed distally up to a maximal protrusion length. Needle 130 is also coupled to a tension spring (not shown) provided in handle assembly 120 so that upon release of needle actuator 122, needle 130 can spring back to its retracted state. Therefore, upon a single full press of needle actuator 122, needle 130 is pushed distally to a maximal protrusion length taking with it pusher 155 and linear ratchet 160 to substantially same travel, using tooth 135. Upon release of needle actuator 122 (or automatically, optionally upon a full press thereof), needle 130 is retracted to its previous position yet pusher 155 and linear ratchet 160 are kept in place being restricted from proximal progression, while needle 130 is allowed to move proximally relative to it, such that tooth 135 is shifted from a particular indentation 162 to a proximally adjacent indentation. Each segmented travel of pusher 155, following a single pressing-releasing sequence of needle actuator 122, is sized to push distally tissue connecting elements 153 in a length of a single tissue connecting element. Moreover, each single sequence of pressing-releasing needle actuator 122 will dispense a single distal-most tissue connecting element 154 to a partial or full protrusion through first jaw distal end 114 or/and opening 115, while needle 130 is in a retracted position exposing it fully or partially.

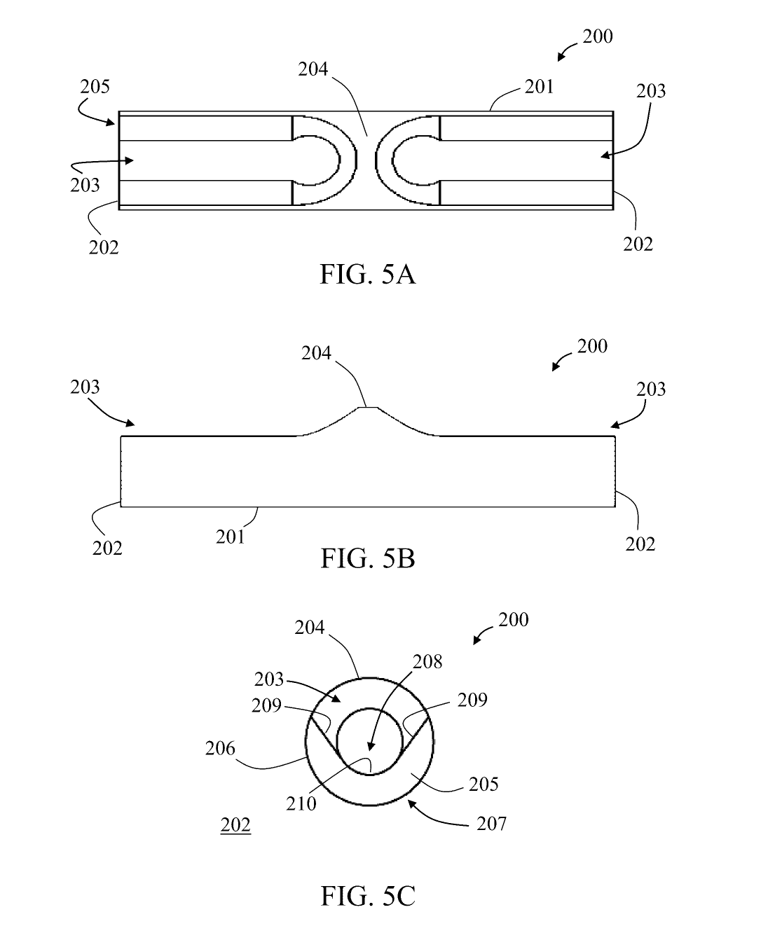

Referring back to FIG. 3B, second jaw 113 includes a holder 125 included or provided in or as part of jaw opening 117, and is configured for coupling with distal-most tissue connecting element 154, or/and arresting it to second jaw 113, when second jaw 113 is at the closed position and if the distal-most tissue connecting element 154 protrudes partially through first jaw distal end 114 while needle 130 is at the retracted position (as shown in FIG. 5F). In some embodiments, needle 130, tissue connecting elements 153 or/and holder 125 and jaw opening 117 are configured such that holder 125 cannot couple to and lock onto needle 130 yet can hold firmly a single tissue connecting element 153. Optionally, the needle 130, at least at its distal end portion, is rigid or/and having a smooth external surface so its slips through the holder when in motion. Tissue connecting elements 153, on the other hand, are slightly compressible or/and having a coarser external surface so holder 125 can hold it in place when exposed by needle 130.

In some embodiments, a restraint is provided (not shown), optionally in shaft lumen 103 adjacent first jaw distal end 114, for restraining a tissue connecting element 153 from passing distally therethrough—up to a maximal restraining force. Optionally, the portion of needle lumen 131 adjacent needle distal tip 132 is slightly narrower or/and coarser to function as a restraint. The restraint can be located such that upon partial protrusion of distal-most tissue connecting element 154 through shaft distal end 114, the restraint restrains a trailing tissue connecting element 153 immediately following the distal-most tissue connecting element 154. The holder is configured to apply a coupling force greater than the maximal restraining force so that distal-most tissue connecting element 154 is releasable from the restraint upon the second jaw 113 shifting from the closed position to the opened position with the holder 125 coupled to distal-most tissue connecting element 154. Optionally (not shown) holder 125 includes a holder actuator configured for selectively operating a holding member between a holding configuration and a releasing configuration automatically or manually.

Throughout the delivery phase, thread 151 is optionally tensioned with a selected (in progress or pre-determinately) tensioning force in order to avoid tangling. In some embodiments, thread 151 tensioning is made between stop element 152 and a point at the thread proximally to the partitionable (separable) shell of tissue connecting elements 153. As shown in FIG. 3G, handle assembly 120 houses a thread tensioner 170 for tensioning thread 151 between the tensioner and stop element 152. Tensioner 170 is fixedly connected to and allowed to swivel with a bobbin 172 using a bobbin connection 171, Thread 151 is wound around bobbin 172 to a coil 173 of selected length. Tensioner 170 optionally includes a spring, such as a recoil spring, for continuously applying tensioning force to thread 151 while allowing extension of the thread to a longer length between bobbin 173 and tensioner 170 and stop element 152 when thread 151 is pulled distally under a pulling force greater than a maximal tensioning force by tensioner 170. A tensioner lock 123 (shown partially in FIG. 3A) may be provided for locking thread 151 at a selected extension length between stop element 152 and tensioner 170.

Reference is made to FIGS. 4A-40, which schematically illustrate side cut views of first jaw 112 connected to shaft 101, which comprises an exemplary needle track 110. Needle track 110 is a portion in shaft lumen 103 provided in first jaw 112 in approximation to distal end 114 thereof, FIG. 4A shows first jaw 112 in a possible disassembled state in case needle 30 is absent (shown for exemplary illustrative purpose only), while FIGS. 4B and 40 show needle 130 at its fully retracted and fully protruded positions, respectively. Needle 130 is slidable within shaft lumen 103 between a protruding position, in which needle distal tip 132 protrudes through first jaw distal end 114 at a maximal protrusion length, and a retracted position. Track 110 is shaped in a selected route for accommodating the needle moving therein in a selected direction. Track 110 is optionally curved such that needle 130 passing distally therethrough angles towards second jaw 113 (not shown in FIGS. 4A-4C). At the protruded position, needle 130 is angled at an acute angle relative to a longitudinal axis x of shaft 101, Needle 130 includes a flexible portion 134 for allowing adaption to the curved track 110 when passing therethrough, and optionally includes a number of proximate transverse cuts. Optionally and additionally, needle 130 includes a rigid portion 135 distal and adjacent the flexible portion 134. Rigid portion may be needed for facilitating or improving tissue penetrability of the needle with needle distal tip 132. Rigid portion 135 has optionally a length and a shape matching a length or/and a shape of track 110.

In some embodiments, tissue connecting elements provided in a body tissue connecting device, or/and a delivery device thereof, are not in a form of a completely close tube. In some such embodiments, compacting two adjacent tissue connecting elements, by adjoining a contact surface in adjacent opposing side of each tissue connecting element, will result in forming a gap therebetween, along portion of the circumference around each contact surface. Maximizing the contact surface areas of adjacent tissue connecting elements optionally corresponds to maximal alignment of the lumenal areas of the adjacent tissue connecting elements. Optionally, in order to avoid non-coincidence of each two adjacent tissue connecting elements, when compacted one against the other, the area of each contact surface is at least half the cross section area of a tube having inner diameter and outer diameter same as of said tubular wall.

FIGS. 5A-5C schematically illustrate different views of exemplary embodiments of a tissue connecting element 200. In some embodiments, tissue connecting element 200 is configured to be strung and freely slide on a thread and is sized and shaped to pass through a puncture in a body tissue wall (optionally being equal to or less than 3 mm, optionally equal to or less than 1 mm, in outer diameter) when aligned towards the puncture, and to revolve and press against the body tissue wall when pulled back via the thread. Tissue connecting element 200 is optionally elastic, semi-elastic or/and bendable. Optionally, tissue connecting element 200 is made of medical or implant grade stainless steel or polymeric material such as PEEK.

Tissue connecting element 200 includes a tubular wall 201 extending between opposing ends 202, each of end merges with a corresponding longitudinal opening 203. Longitudinal openings 203 oppose each other and are separated with a strip 204. Each opposing end 202 includes a contact surface 205 having an area being at least half a cross section area of a (virtual) tube having inner diameter and outer diameter same as of tubular wall 201. In some embodiments, tissue connecting element 200 is configured and arranged such that upon the thread being pulled back, the thread portion located therein shifts from extending between opposing ends 202 to wrapping around strip 204.

In some embodiments, a perimeter 206 enclosing contact surface 205 includes an outer part 207 and an inner part 208. Perimeter outer part 207 is optionally greater than half a circumference of the (virtual) tube. In some embodiments, perimeter inner part 208 includes two symmetrically opposing tangential lines 209 touching both ends of a curve 210 having an inner diameter same as the (virtual) tube inner diameter.

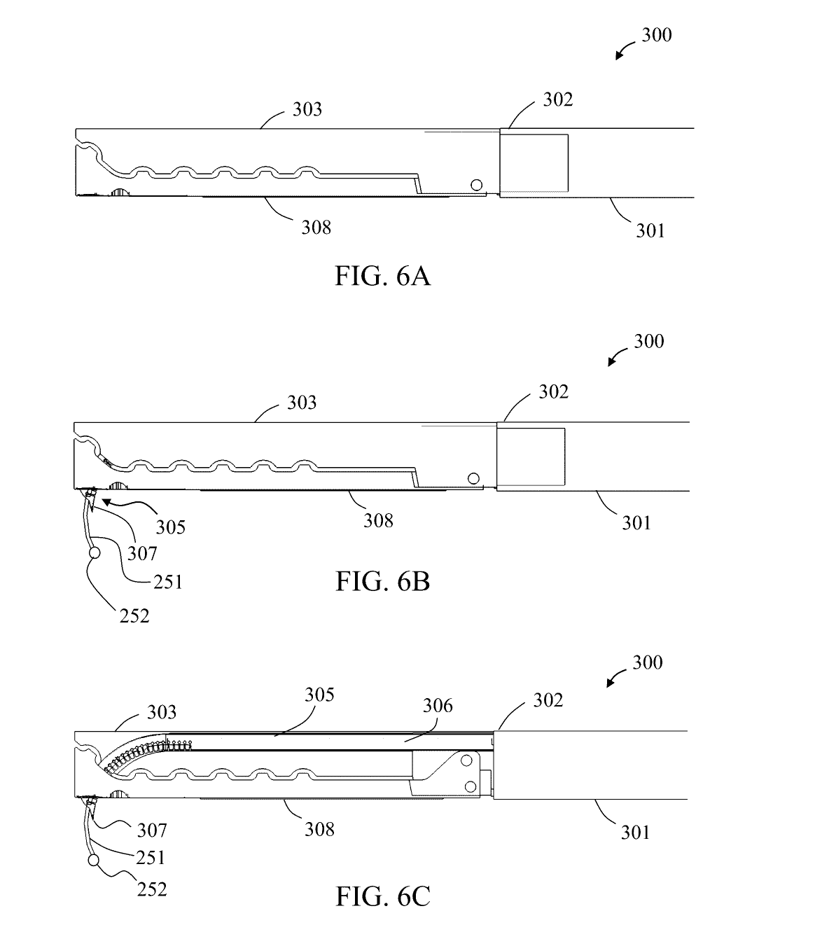

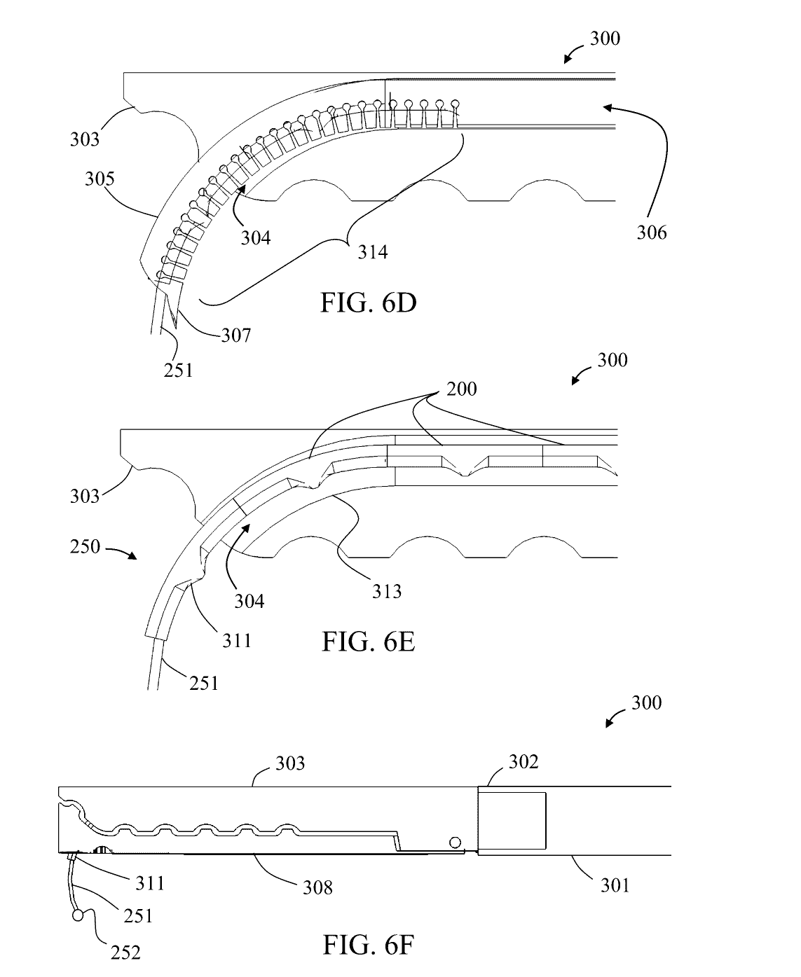

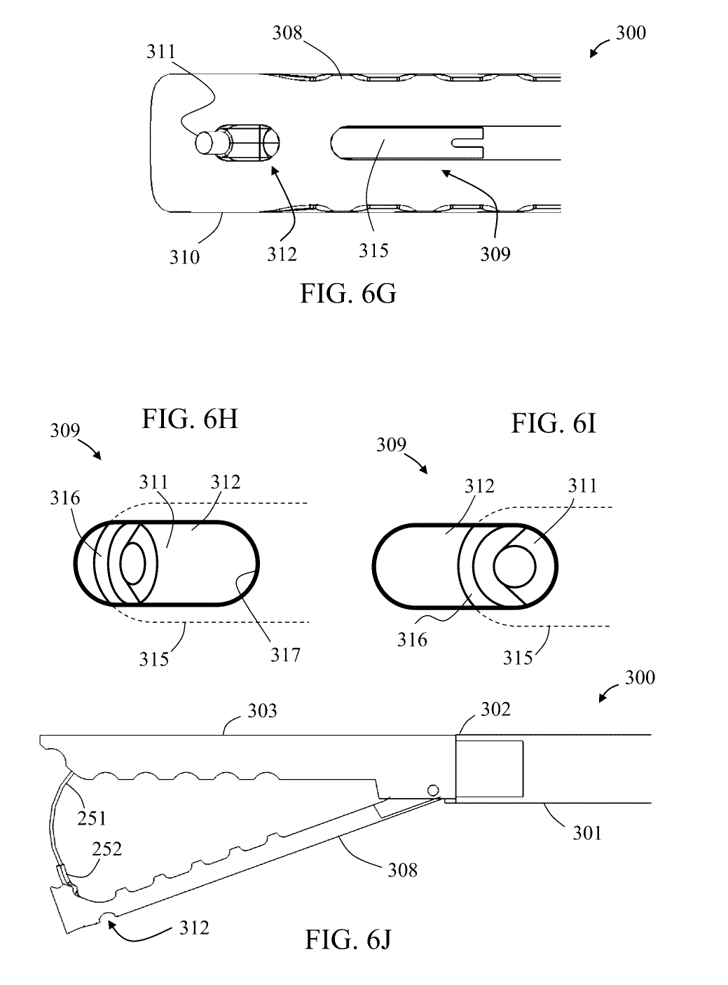

FIGS. 6A-6J schematically illustrate exemplary embodiments of an exemplary delivery device 300, for delivering a body tissue connecting device 250 that includes a plurality of tissue connecting members, optionally, each being the same as tissue connecting element 200. Delivery device 300 includes a shaft 301 connected at a distal end 302 thereof to a first jaw 303, both sharing a shaft lumen extending lengthwise therein and opened at a distal end 304 of first jaw 303.

A needle 305, which includes a needle lumen 306 and a needle distal tip 307, extends inside the shaft lumen. Needle lumen 305 is sized to house a plurality of tissue connecting elements 200 strung on a thread 251 provided and extending therein. Thread 251 is connected at a distal end thereof to a stop element 252, configured for supporting compaction of the tissue connecting members 200 when pushed together against it, optionally using a pusher (not shown), FIG. 6E illustrates a number of tissue connecting elements 200 compacted one to the other and against stop 252, virtually within needle 305 (not explicitly visible, rather transparent in this figure, for exemplary illustrative purposes).

Needle 305 is slidable in needle lumen 306 to a protruding position (as shown in FIGS. 6B-6D), in which needle distal tip 307 protrudes through first jaw distal end 304 at a pre-determined maximal protrusion length, and to a retracted position (shown in FIGS. 6A and 6J).

A second jaw 308 is pivotally connected at shaft distal end 302 and is linked to and actuatable by a jaw actuator between a closed position (shown in FIGS. 6A-60 and 6F), whereby second jaw 308 is in close approximation with first jaw 303, and an opened position (shown in FIG. 6J), whereby second jaw 308 is pivoted away from first jaw 303. The jaws are sized, shaped and arranged to grasp a body tissue having a thickness within a range of pre-determined allowed thickness values. An exemplary range of pre-determined allowed thickness values is between about 0.1 mm and about 10 mm.

In some embodiments and as shown, especially in FIG. 6D, needle distal tip 307 is pointed so as to penetrate through a body tissue having, or compressible to, a thickness equal to or less than said pre-determined maximal protrusion length. As shown, needle distal tip 307 projects from a lower part of needle distal end at which an upper part of 305 needle distal end is substantially flat.

In some embodiments, the shaft lumen portion extending across within first jaw 303 includes a curved section 313 (shown in FIG. 6E) to thereby angularly force needle 305 therethrough such that needle distal 307 tip will angle towards second jaw 308. Optionally, needle distal tip 307 angles to an acute angle relative to a longitudinal axis of shaft 301. Needle 305 includes a flexible portion 314 with a number of proximate transverse cuts. Needle 305 may also include a rigid portion distal to and adjacent flexible portion 314, which may have length and shape matching a length or/and a shape of curved section 313.

Delivery device 300 optionally includes a thread tensioner configured to apply tension to thread 251 extending between the tensioner and stop element 252. The thread tensioner may include a spring configured to apply a continuous tensioning force to thread 251 while allowing or forcing it to extend to a longer length between tensioner and stop element 252 when thread 251 is pulled distally under a pulling force greater than a maximal tensioning force applied by the thread tensioner. A tensioner lock may be provided for locking thread 251 at a selected extension length between stop element 252 and the thread tensioner.

A holder mechanism 309 (shown in FIG. 6G and schematically illustrated in FIGS. 6H and 61) is provided in a distal end 310 of second jaw 308 and configured to arrest a distal-most tissue connecting element 311 to second jaw 308 (FIG. 6I) when second jaw 308 is in the closed position and when distal-most tissue connecting element 311 partially protrudes from first jaw distal end 304 while needle 305 is in the retracted position (as shown in FIG. 6F).

An entrapment opening 312 is provided in second jaw 308 and is sized to allow needle 305 to pass therethrough. Optionally, upon second jaw 308 grasping the body tissue, the needle distal tip 307, when protruded to the maximal protrusion length, passes through entrapment opening 312 while penetrating through the body tissue. Entrapment opening 312 is sized to allow needle 305 to pass therethrough via an acute angle relative to second jaw 308, as shown.

Delivery device 300 includes a restraint or a restraining means configured to restrain any of tissue connecting elements 200 from passing distally therethrough, when they are subjected to a pushing force less than or equal to a maximal restraining force. The tissue connecting elements 200 are compactable to a selected configuration when pressed against the restraint with the pushing force when it is less than or equal to this maximal restraining force. Curved section 313 in needle lumen 306 and surrounding wall of needle 305 may serve as restraining means, whereby any of tissue connecting elements 200 forced therethrough will bend in conformity thereto. Further restraining facilitators may be in the form of bulges or/and slits 314 across surrounding wall of needle 305, along a section thereof, to thereby increase resistance to motion of any tissue connecting element 200 forced thereacross.

Optionally, holder mechanism 309 is configured to apply a coupling force greater than the maximal restraining force so that distal-most tissue connecting element 311 is releasable from the restraint upon second jaw 308 shifting from the closed position to the opened position with distal-most tissue connecting element 311 being arrested thereto (FIG. 6J).

Holder mechanism 309 may include a holder actuator configured to selectively operate a holding member 315 between a holding configuration (FIG. 6I), in a tissue connecting element 200 is arrestable to second jaw 308, and a releasing configuration (FIG. 6H), in which a previously arrested tissue connecting member 200 to second jaw 308 may be released therefrom.

Holding member 315 includes a gripping portion 316 slidable across entrapment opening 312 between the releasing configuration, in which gripping portion 316 is positioned distally beyond entrapment opening 312, and the holding configuration, in which gripping portion 316 approximates entrapment opening 312 proximal side 317 in a distance equal to or less than outer diameter of needle 305. Optionally, gripping portion 316 approximates entrapment opening 312 proximal side 317 to a distance equal to or less than outer diameter of distal-most tissue connecting element 311, to thereby arrest it to second jaw 308 upon withdrawal of needle 305 to the shaft lumen.

In some embodiments, holder mechanism 309 is configured to force distal-most tissue connecting element 311 to align normally to second jaw 308, as shown in the shift of tissue connecting element 200 between its positioning in FIG. 6H to its positioning in FIG. 6I, optionally upon withdrawal of needle 305 to the shaft lumen and to arrest it to second jaw 308.

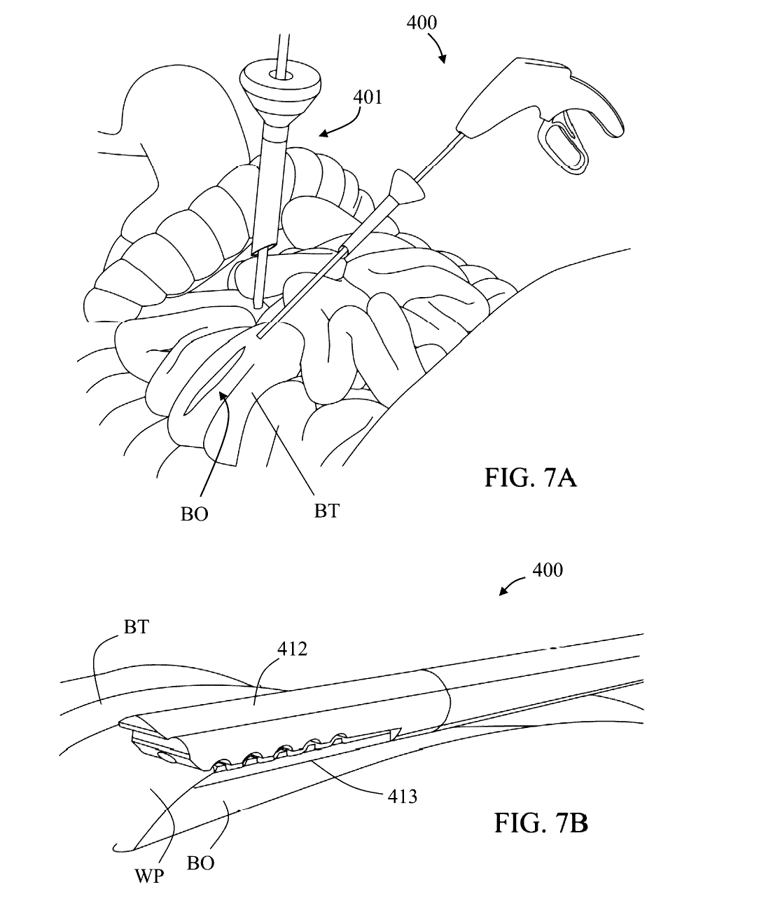

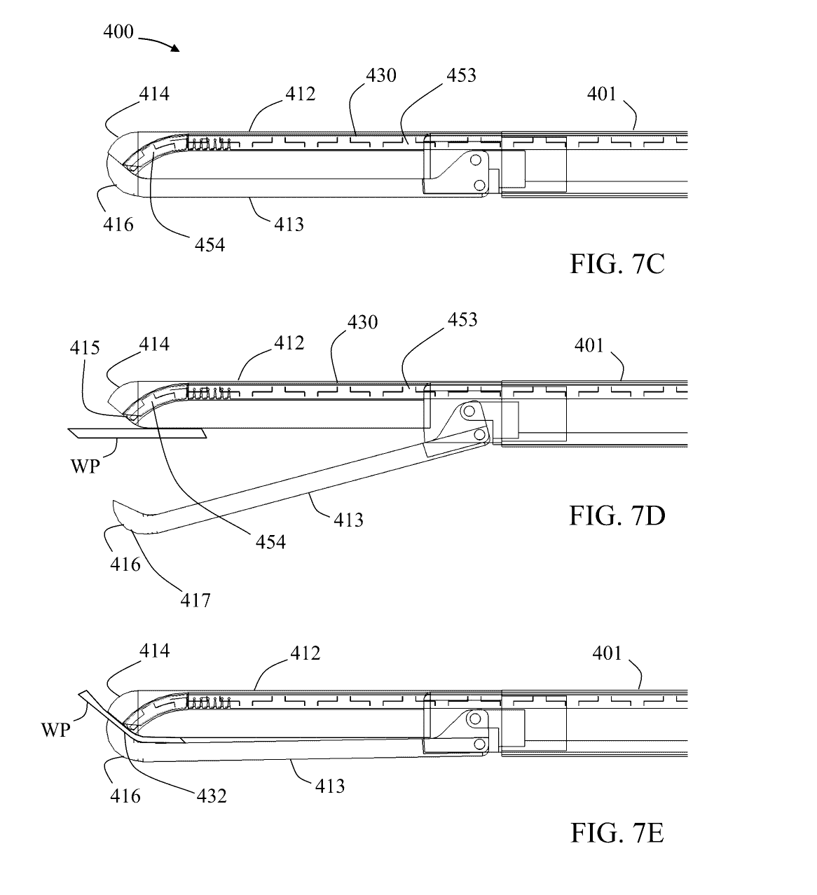

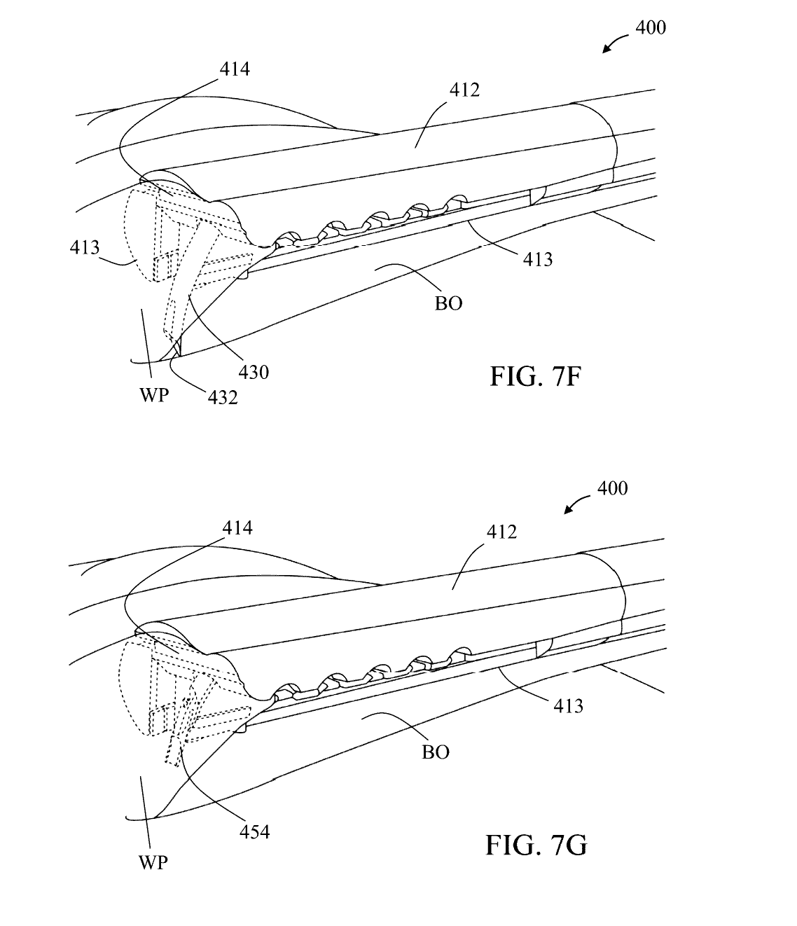

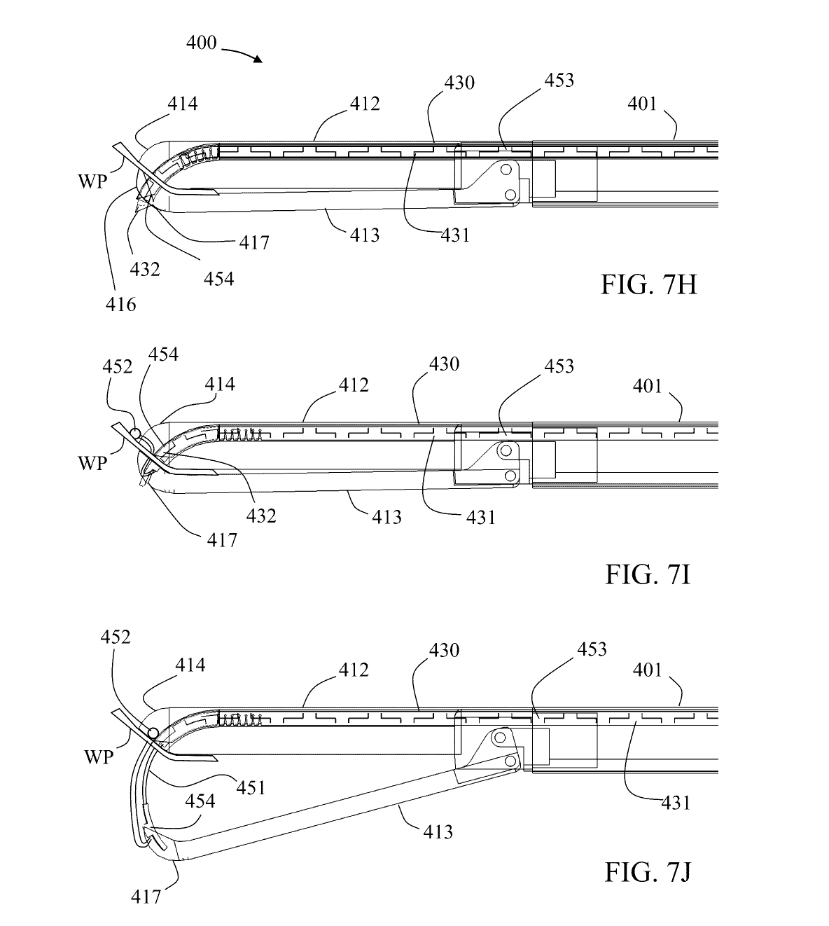

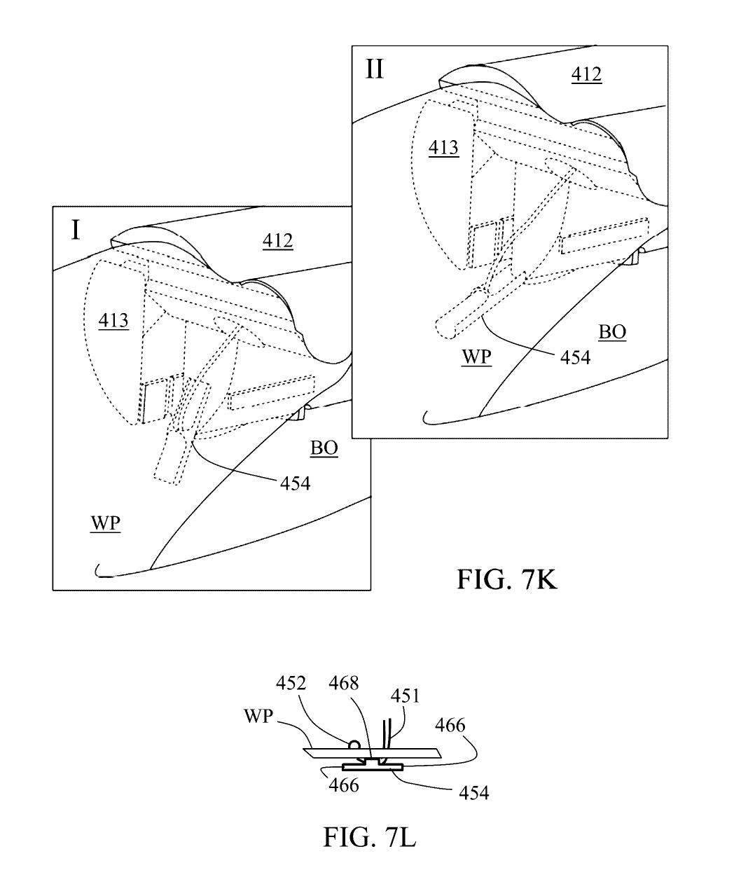

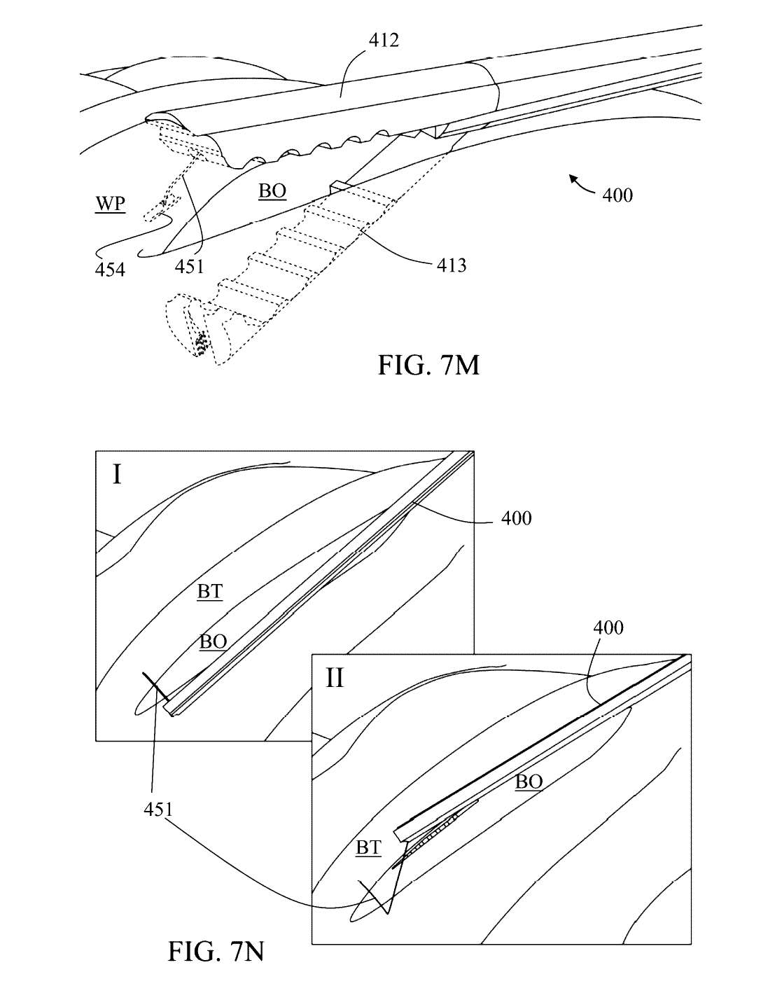

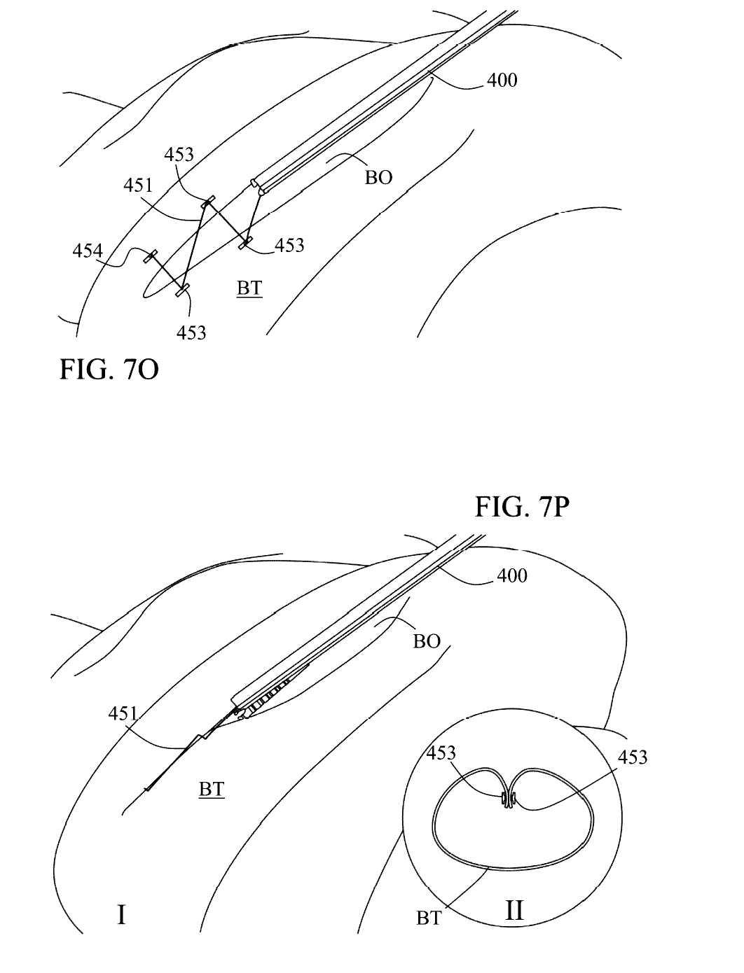

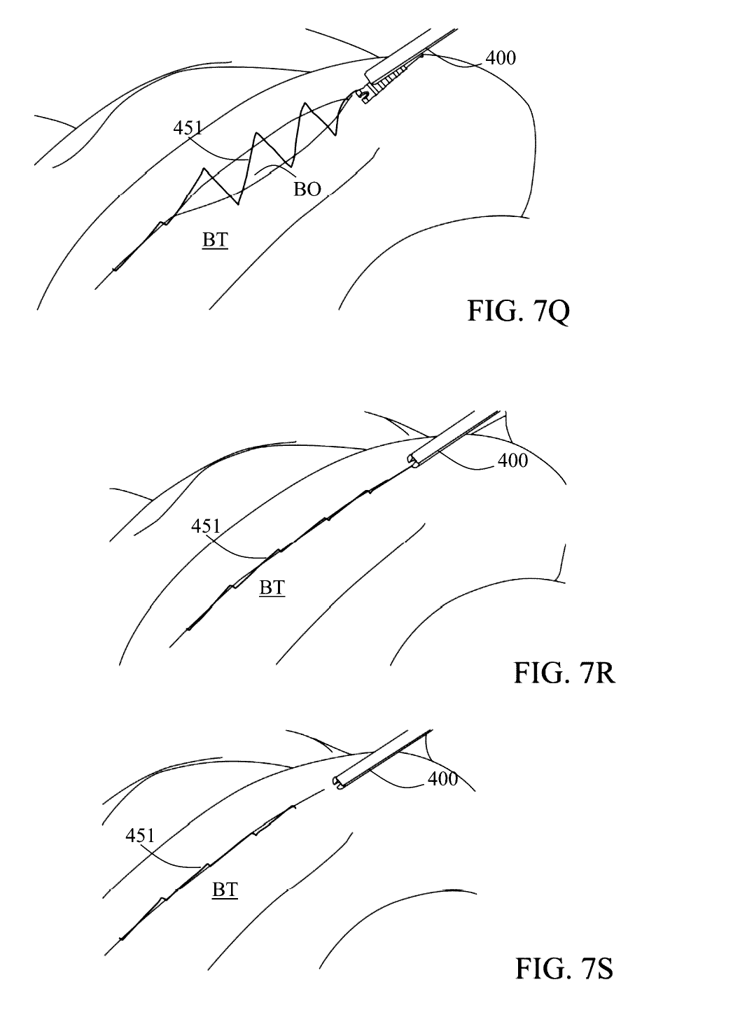

Reference is now made to FIGS. 7A-7SG, which schematically illustrate exemplary embodiments of possible scenarios in a method for deploying tissue connecting elements 453, including a distal-most tissue connecting element 454, in a surgical process of closing a body opening BO adjacent a body tissue BT, using delivery device 400. Device 400 is provided, optionally readily, with a plurality of tissue connecting elements 453 strung on a thread 451, proximally to a stop element 452. Thread 451 is tensioned, optionally readily tensioned, between a stop element 452 and a tensioner. Device 400 may be functional or/and designed similarly or identically to any of delivery devices 40, 100, and 300. Tissue connecting elements 453 may be designed, shaped or/and structured similarly or identically to any of tissue connecting elements 13, 23, 33, 43, 53, 153, and 200. FIG. 7C may represent a common scenario after device 400 is unboxed or after it had been prepared for surgery, prior to use.

FIG. 7A shows a laparoscopic surgical intervention (not showing abdomen and other tissues for illustrative-demonstrative purpose) in which delivery device 400 is positioned via a small diameter trocar (e.g., about 6 mm or less in lumen diameter) towards body opening BO, and monitored using a laparoscope 401.

As shown in FIGS. 7B, 7D and 7E, delivery device 400 is first used to grasp wall portion WP of the body tissue BT adjacent to boundary of body opening BO with a first jaw 412 and a second jaw 413. A medical practitioner using delivery device 400 may apply it to grasp or pinch different wall portions of body tissue BT or elsewhere before, or even without eventually applying it for tissue penetration or/and delivery of any of the tissue connecting elements 453. The jaws may include an upwardly bent portion about first jaw distal end 414 to allow a needle 430 to curve in an acute angle towards second jaw 413, thereby also forcing wall portion WP to deform accordingly between the jaws (as shown in FIG. 7E), In FIGS. 7F, 7G, 7K and 7M a frontal portion of second jaw 413 is made transparent, or/and some parts concealed by wall portion WP of delivery device 400 and of distal-most tissue connecting element 454 are provided in dashed lines, for illustrative-demonstrative purpose of explaining certain functions or mechanisms relevant to the surgical intervention.

As shown in FIGS. 7F and 7H, wall portion WP is penetrated with needle 430 distal tip 432 such that it moves from pointing towards an external surface of wall portion WP (as shown in FIG. 7E) to protruding beyond an internal surface of the wall portion WP (FIG. 7H). Needle 430 is pushed distally to the protruding position, in which needle distal tip 432 protrudes through first jaw distal end 414 at a maximal protrusion length. Also as shown in FIG. 7H, distal-most tissue connecting element 454 is positioned beyond the internal surface of wall portion WP in needle lumen 431.

As shown in FIGS. 7G and 7I, needle 430 is then retracted back away from wall portion WP, such that needle distal tip 432 is back in shaft lumen 430, while distal-most tissue connecting element 454 is kept beyond the internal surface of wall portion WP and exposed following retraction of needle 430.

As shown in FIG. 7J, distal-most tissue connecting element 454 is separated from the partitionable (separable) shell containing the other tissue connecting elements 453, and spacing therefrom, by actuating second jaw 413 to shift from the closed position to the opened position while distal-most tissue connecting element 454 is coupled to the holder and held in a jaw opening 417, while a trailing (i.e., immediately following) tissue connecting element 453 is held back by a restraint restraining means. As a result, wall portion WP is also released from the jaws grasping.

In some embodiments, thread 451 is tensioned continuously with a tensioner/tensioning means installed in delivery device 400 during at least one of the steps of penetrating wall portion WP, positioning distal-most tissue connecting element 454 beyond the internal surface of wall portion WP, retracting needle 430 and separating distal-most tissue connecting element 454 from the other tissue connecting elements 453.

Each tissue connecting element 453 is configured to pass through a puncture in a body tissue wall when aligned towards the puncture and to revolve and press against the body tissue wall when pulled back via the thread. FIG. 7K shows the shift in positioning of distal-most tissue connecting element 454 immediately after it is exposed by needle 430 (FIG. 7K(I)) and after it is pulled back against wall portion WP (FIG. 7K(II)). Once provided completely beyond the internal surface of wall portion WP, distal-most tissue connecting element 454 is released from holder and opening 417, either by pulling it out (e.g., manually) or, in case holder is selectively actuatable, by activating the holder to release mode. By pulling thread 451 proximally, the thread portion provided in distal-most tissue connecting element 454 shifts to wrapping around a strip 468 projecting therefrom followed by the distal-most tissue connecting element 454 revolving and pressing against body tissue wall portion WP at the internal surface thereof, FIG. 7L shows distal-most tissue connecting element 454 following revolving thereof to a parallel orientation with respect to wall portion WP in which it can function as a tissue anchor. While withdrawing delivery device 400 from the first anchored tissue connecting element 454, as shown in FIG. 7M, second jaw 413 is in opened position and the thread tensioner is inactive (e.g., by user preference) or that delivery device 400 is pulled in a force greater than the maximal tensioning force applied by the thread tensioner.

FIGS. 7N-7S shows sequence of steps for completing disposals of a selected number of tissue connecting elements and closing the body opening BO. The above described steps are repeated, each time with a “new” distal-most tissue connecting element consecutive to a “previous” distal-most tissue connecting element, until a selected number of tissue connecting elements 453 is separated from the partitionable (separable) shell or until the partitionable (separable) shell is completely partitioned (separated) into separate tissue connecting elements 453. Each separated tissue connecting element 453 is optionally provided beyond a different body tissue wall portion such that each two consecutive separated tissue connecting elements 453 are located at different sides of the body opening BO. FIG. 7N shows deployment of a second tissue connecting element (FIG. 7N(I)) followed by withdrawal of delivery device 400 and repositioning towards a third deployment place (FIG. 7N(II)). FIG. 7O shows withdrawal of delivery device 400 upon deployment of four tissue connecting elements (appearing partly hidden behind body tissue BT, for exemplary illustrative purposes).

Thread 451 may be forced or allowed (by threat tensioner) to be pulled during the procedure, optionally automatically or/and according to user preference, even before deployment of all selected tissue connecting elements 453, resulting in continuous, yet partial, closing of body opening BO, as show in FIG. 7P(I). Forcing the separated tissue connecting elements 453 together results in an inverted suturing upon closing of the body tissue opening, as shown in FIG. 7P(II).

FIG. 7Q shows completion of deployment of the entire selected number of tissue connecting elements 453. Thread 451 is pulled (FIG. 7R) followed by securing all separated tissue connecting elements to a selected final length of thread 451, which are optionally left implanted in-place in the patient body.

Each of the following terms written in singular grammatical form: ‘a’, ‘an’, and ‘the’, as used herein, means ‘at least one’, or ‘one or more’. Use of the phrase ‘one or more’ herein does not alter this intended meaning of ‘a’, ‘an’, or ‘the’. Accordingly, the terms ‘a’, ‘an’, and ‘the’, as used herein, may also refer to, and encompass, a plurality of the stated entity or object, unless otherwise specifically defined or stated herein, or, unless the context clearly dictates otherwise. For example, the phrases: ‘a unit’, ‘a device’, ‘an assembly’, ‘a mechanism’, ‘a component’, ‘an element’, and ‘a step or procedure’, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: ‘includes’, ‘including’, ‘has’, ‘having’, ‘comprises’, and ‘comprising’, and, their linguistic grammatical variants, derivatives, or/and conjugates, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The phrase ‘consisting essentially of’, as used herein, means that the stated entity or item (system, system unit, system sub-unit, device, assembly, sub-assembly, mechanism, structure, component, element, or, peripheral equipment, utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional ‘feature or characteristic’ being a system unit, system sub-unit, device, assembly, sub-assembly, mechanism, structure, component, or element, or, peripheral equipment, utility, accessory, or material, step or procedure, sub-step or sub-procedure), but only if each such additional ‘feature or characteristic’ does not materially alter the basic novel and inventive characteristics or special technical features, of the claimed entity or item.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, from 1 to 4′, from 1 to 5′, ‘from 2 to 4’, ‘from 2 to 6’, ‘from 3 to 6’, etc., and individual numerical values, such as ‘1’, ‘1.3’, ‘2’, 2.8, ‘3’. ‘3.5’, ‘4’, ‘4.6’. ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase in a range of between about a first numerical value and about a second numerical value′, is considered equivalent to, and meaning the same as, the phrase ‘in a range of from about a first numerical value to about a second numerical value’, and, thus, the two equivalently meaning phrases may be used interchangeably. For example, for stating or describing the numerical range of room temperature, the phrase ‘room temperature refers to a temperature in a range of between about 20° C. and about 25° C.’, and is considered equivalent to, and meaning the same as, the phrase ‘room temperature refers to a temperature in a range of from about 20° C. to about 25° C.’.

The term ‘about’, as used herein, refers to ±10% of the stated numerical value. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1.-57. (canceled)

58. A method for closing an opening in body tissue, the method comprising:
providing a needle comprising a needle distal tip and a needle lumen opened at said distal needle tip, said needle lumen housing a thread, fixedly connected at a distal end thereof to a stop element, and a partitionable shell comprising a plurality of elongated tissue connecting elements strung on said thread proximally to said stop element and compactable along said thread towards said stop element, wherein each of said tissue connecting elements includes a tubular wall extending between opposing ends, each of said opposing ends merges with a corresponding longitudinal opening, said longitudinal openings being opposed to each other and separated with a strip;
tensioning said thread between said stop element and a point at said thread proximally to said partitionable shell, via applying a tensioning force to said thread;
penetrating a wall portion of body tissue adjacent the body tissue opening with said needle distal tip such that said needle distal tip moves from pointing towards an external surface of said wall portion to protruding beyond an internal surface of said wall portion;
positioning said tissue connecting element located distal-most along said partionable shell beyond said internal surface of said wall portion in said needle lumen;
retracting said needle back from said wall portion while holding said distal-most tissue connecting element beyond said internal surface of said wall portion such that said needle exposes said distal-most tissue connecting element; and
separating said distal-most tissue connecting element from said partionable shell and spacing therebetween.

59. The method of claim 58, comprising:
pulling said thread proximally such that a thread portion located within said distal-most tissue connecting element shifts from extending between said opposing ends to wrapping around said strip followed by said distal-most tissue connecting element revolving and pressing against said wall portion at said internal surface thereof.

60. The method of claim 58, wherein said tensioning of said thread is continuously performed during said penetrating, said positioning, said retracting and said separating, and includes, or is included in, said pulling of said thread, such that said tensioning force facilitates or contributes to said pulling.

61. The method of claim 58, being repeated, each time with a new distal-most tissue connecting element consecutive to a previous distal-most tissue connecting element, until a selected number of said tissue connecting elements is separated from said partitionable shell, or until said partitionable shell is completely partitioned such that said tissue connecting elements become separated from each other.

62. The method of claim 61, wherein each of said separated tissue connecting elements is provided beyond a different wall portion of the body tissue such that at least two consecutive separated tissue connecting elements are located at different sides of the body tissue opening.

63. The method of claim 62, comprising:
forcing said separated tissue connecting elements together against said stop element such that said separated tissue connecting elements converge along a final length of said thread while the body tissue opening substantially closes; and
securing said separated tissue connecting elements to said final length of said thread.

64. The method of claim 63, wherein said forcing of said separated tissue connecting elements together results in an inverted suturing upon closing of the body tissue opening.

65. The method of claim 61, comprising, between two of said repetitions:
extending said thread to a selected extension length with a pulling force greater than said tensioning force; and
locking said thread at said selected extension length.

66. The method of claim 58, wherein said needle is provided in a shaft lumen of a shaft connected to an first jaw with a distal end thereof, said shaft lumen extending lengthwise therein and opened at said first jaw distal end;
wherein said penetrating of said wall portion includes pushing said needle distally in said shaft lumen to a protruding position, in which said needle distal tip protrudes through said first jaw distal end at a maximal protrusion length; and
wherein said retracting of said needle back from said wall portion includes pulling said needle proximally to a retracted position, in which said needle distal tip is inside said shaft lumen.

67. The method of claim 66, wherein a second jaw is connected at distal end of said shaft or/and to said first jaw distal end configured to selectively actuate between a closed position and an opened position, said arm member includes a holder mechanism configured to arrest said distal-most tissue connecting element to said second jaw when said second jaw is at said closed position and when said distal-most tissue connecting element partially protrudes through said shaft distal end while said needle is in said retracted position.

68. The method of claim 67, wherein said retracting said needle includes applying said holder mechanism to arrest said distal-most tissue connecting element to said second jaw.

69. The method of claim 67, wherein said separating said distal-most tissue connecting element from said partitionable shell includes actuating said second jaw to shift from said closed position to said opened position.

70. The method of claim 67, wherein said jaws are sized, shaped and arranged so as to grasp the body tissue having a thickness within a range of pre-determined allowed thickness values.

71. The method of claim 70, further comprising grasping said wall portion of the body tissue with said jaws.

72. The method of claim 71, wherein said separating said distal-most tissue connecting element from said partitionable shell includes releasing said wall portion from said grasping jaws.

73. The method of claim 67, further comprising activating said holder mechanism to release said distal-most tissue connecting element when said distal-most tissue connecting element extends completely beyond said internal surface of said wall portion.
